(19) United States
(12) Patent Application Publication
(10) Pub. No.: US 2026/0158296 A1
(43) Pub. Date: Jun. 11, 2026

(54) EXTRACORPOREAL THERAPEUTIC ULTRASOUND FOR VASCULAR CALCIUM TREATMENT

(71) Applicant: Vibrato Medical, Inc., Newport Beach, CA (US)

(72) Inventors: Babak Nazer, Newport Beach, CA (US); James Gessert, Newport Beach, CA (US); Juliana Elstad, Newport Beach, CA (US)

(21) Appl. No.: 18/971,691

(22) Filed: Dec. 6, 2024

(51) Int. Cl. A61N 7/00

(52) U.S. Cl. CPC A61N 7/00; A61N 2007/0004; A61N 2007/0039

(57) ABSTRACT

Systems, methods, and devices of the present disclosure can facilitate treatment of calcified vascular tissue of a patient. Treatment can include providing a therapeutic ultrasound (TUS) device configured to generate a TUS acoustic filed and providing a diagnostic imaging ultrasound transducer coupled to or used in conjunction with the TUS device. The imaging transducer may be configured to display a location of the TUS acoustic field superimposed on a diagnostic ultrasound image. Treatment can include focusing a TUS acoustic field of the TUS transducer onto a region of vascular calcium within a vessel of the patient below the skin surface. Treatment can include generating a TUS energy waveform, the waveform comprising a therapeutically effective amount of TUS energy, and directing the therapeutically effective amount of TUS energy toward the region of vascular calcium to stimulate cavitation and shear stress and to remove or soften a calcium deposit within the region of vascular calcium.

### INCORPORATION BY REFERENCE TO ANY PRIORITY APPLICATIONS

Any and all applications for which a foreign or domestic priority claim is identified in the Application Data Sheet as filed with the present application are hereby incorporated herein by reference under 37 CFR 1.57.

### FIELD

The present application relates to therapeutic ultrasound (TUS) and peripheral vascular disease (PVD), including peripheral arterial disease (PAD).

### BACKGROUND

PAD is a highly prevalent condition, affecting approximately 202 million people worldwide and 8.5 million people in the USA. Of these, approximately one-third are symptomatic with claudication (lower extremity muscle pain with walking that limits activity), and 2-3% progress to critical limb ischemia (CLI), which is a highly morbid condition associated with resting pain, skin ulcers, and gangrene, often requiring amputation.

### SUMMARY

Systems and methods as disclosed herein can offer in some embodiments a non-invasive, non-surgical, outpatient treatment for peripheral vascular disease (PVD), including PAD and a variety of other indications, which can be used throughout the lifespan of the patient to treat symptoms and prevent disease recurrence. Some embodiments can advantageously reduce the incidence of amputations, hospitalizations, and other complications of advanced PAD, adding substantial value for patients, physicians, providers, and payers alike. Many PAD patients suffer from immobility due to claudication, foot ulcers, and other co-morbidities, making wearable, home, night-time use well-suited in some cases. Some embodiments can also be used as an adjunctive treatment to invasive procedures, augmenting the capacity of the limited number of physicians trained in endovascular techniques. Some embodiments alternatively allow high risk patients to avoid surgery and reduce risk from surgical or catheter-based procedures, which even when feasible, can have a 20-40% failure rate in some cases.

Systems and methods can include wearable, non-invasive ultrasound modalities for treating a variety of medical conditions. These conditions include but are not limited to peripheral vascular disease (including PAD), and venous disease such as venous insufficiency. Conventional TUS devices used for physical therapy, CAD, and other medical indications are configured with the sole, supposed goal of promoting vasodilation and increased blood flow, although evidence for increased blood flow and the mechanisms of these effects have so far not yet been described. In contrast, in some embodiments, systems and methods as disclosed herein can advantageously promote revascularization of blood vessels afflicted with vascular calcium and/or condition said vascular calcium to permit use of invasive PAD interventions, such as catheter-based revascularization. Systems and methods disclosed herein may promote revascularization through one, two, or more ultrasound-mediated mechanisms. Not to be limited by theory, these mechanisms can include, for example, revascularization and/or conditioning of vascular calcium through shear stress and cavitation.

Various implementations of systems, methods, and devices within the scope of the appended claims each have several aspects, no single one of which is solely responsible for the desirable attributes described herein. Without limiting the scope of the appended claims, the description below describes some prominent features.

Details of one or more implementations of the subject matter described in this specification are set forth in the accompanying drawings and the description below. Other features, aspects, and advantages will become apparent from the description, the drawings, and the claims. Note that relative dimensions of the following figures may not be drawn to scale.

In some implementations, the present disclosure relates to a method of treating calcified vascular tissue, comprising: providing a therapeutic ultrasound device configured to generate a therapeutic ultrasound acoustic field; providing a diagnostic imaging ultrasound transducer coupled to or used in conjunction with the therapeutic ultrasound device and configured to display a location of the therapeutic ultrasound acoustic field superimposed on the diagnostic ultrasound image; positioning the ultrasound transducer proximate a skin surface of a patient; focusing an acoustic field of the therapeutic ultrasound transducer onto a region of vascular calcium within a vessel of the patient below the skin surface; generating a therapeutic ultrasound energy waveform, the therapeutic ultrasound energy waveform comprising a therapeutically effective amount of therapeutic ultrasound energy; and directing the therapeutically effective amount of therapeutic ultrasonic energy toward the region of vascular calcium to stimulate cavitation and shear stress and to remove or soften a calcium deposit within the region of vascular calcium.

In some implementations, the therapeutic ultrasonic energy has a frequency of 400 kHz to 5 MHz.

In some implementations, the therapeutic ultrasonic energy has a pulse duration of 5 μs to 100 ms.

In some implementations, the therapeutic ultrasonic energy has a pulse repetition frequency of 0.1 Hz to 5 kHz.

In some implementations, the therapeutic ultrasonic energy has a peak negative pressure of 2 MPa to 24 MPa.

In some implementations, the array of therapeutic ultrasound transducers is a linear array.

In some implementations, the therapeutic ultrasound transducer is geometrically focused, and/or electronically focused, and/or comprises electronic and/or mechanical beam steering.

In some implementations, the therapeutic ultrasound transducer is shaped to treat a longitudinal segment of the vessel.

In some implementations, the longitudinal segment of the vessel is up to 20 cm long.

In some implementations, directing the therapeutically effective amount of therapeutic ultrasonic energy toward the region of vascular calcium comprises directing the therapeutically effective amount of therapeutic ultrasonic energy toward the region of vascular calcium for 1 to 120 minutes.

In some implementations, directing the therapeutically effective amount of therapeutic ultrasonic energy toward the region of vascular calcium comprises directing the therapeutically effective amount of therapeutic ultrasonic energy toward the region of vascular calcium for a plurality of intervals.

In some implementations, the method further comprises providing an imaging transducer configured to generate an image of the vessel.

In some implementations, the method further comprises generating an image of the region of vascular calcium within the vessel.

In some implementations, the method further comprises displaying the image on a display coupled to the therapeutic ultrasound device.

In some implementations, the therapeutic ultrasound device is further configured to automatically position the ultrasound transducer based on the image.

In some implementations, the therapeutic ultrasound device is further configured to automatically focus the acoustic field of the ultrasound transducer onto the region of vascular calcium based on the image.

In some implementations, the method further comprises providing a microbubble solution to the vessel.

In some implementations, the vessel comprises an iliac artery, a common femoral artery, a superficial femoral artery, a popliteal artery, an anterior tibial artery, a posterior tibial artery, a subclavian artery, an axillary artery, a brachial artery, a radial artery, an ulnar artery, a renal artery, a carotid artery, a celiac trunk/artery, a superior mesenteric artery, or an inferior mesenteric artery.

In some implementations, the region of vascular calcium comprises the dorsalis pedis, the posterior tibial, or the pedal arches.

In some implementations, the method further comprises performing a secondary therapy on the vessel after therapeutic ultrasound.

In some implementations, the secondary therapy comprises stenting, drug-coated balloon angioplasty, balloon angioplasty, shockwave atherectomy, orbital atherectomy, or otherwise removing calcium from the vessel.

In some implementations, the method further comprises identifying fluid flow within the vessel.

### BRIEF DESCRIPTION OF THE DRAWINGS

Example features of the present disclosure, its nature and various advantages will be apparent from the accompanying drawings and the following detailed description of various implementations. Non-limiting and non-exhaustive implementations are described with reference to the accompanying drawings, wherein like labels or reference numbers refer to like parts throughout the various views unless otherwise specified. The sizes and relative positions of elements in the drawings are not necessarily drawn to scale. For example, the shapes of various elements may be selected, enlarged, and positioned to improve drawing legibility. The particular shapes of the elements as drawn have been selected for ease of recognition in the drawings.

FIGS. 1 and 2 illustrate potential mechanisms of action of a TUS wearable device.

FIG. 3 illustrates sample waveforms of TUS and SWT, respectively.

FIG. 4 illustrates a non-limiting embodiment of a single-element transducer.

FIG. 5 illustrates a non-limiting embodiment of an arrangement of transducers.

FIGS. 6A-6D schematically illustrate non-limiting embodiments of the TUS wearable device and non-limiting embodiments of securing the device to a subject for treating PAD. Also illustrated are various non-limiting arterial vessels that may be involved in PAD and treated using systems and methods disclosed herein, also in FIGS. 6E-6F.

FIGS. 7A-7D schematically illustrate non-limiting positions of an ultrasound transducer and/or array adjacent the skin surface of an anatomical target location.

FIG. 8 illustrates a diagram illustrating schematically a network of non-limiting examples of devices that can communicate with the TUS wearable device disclosed herein.

FIG. 9 is a block diagram illustrating an example computing device.

FIGS. 10A-10B illustrate an example embodiment of an ultrasound generator and patient interface, respectively.

FIG. 10C illustrates an example embodiment of a TUS device.

FIG. 11 illustrates an example implementation of a TUS system.

FIG. 12 illustrates an example implementation of a TUS system.

FIG. 13 illustrates an example process of performing therapeutic ultrasound using the TUS wearable device.

### DETAILED DESCRIPTION

The drawings and the associated descriptions are provided to illustrate embodiments of the disclosure and not to limit the scope of the claims.

Although certain embodiments and examples are described below, those of skill in the art will appreciate that the disclosure extends beyond the specifically disclosed embodiments and/or uses, and obvious modifications and equivalents thereof based on the disclosure herein. Thus, it is intended that the scope of the disclosure herein disclosed should not be limited by any particular embodiments described below.

Disclosed herein are systems and methods including wearable, non-invasive ultrasound modalities for treating a variety of medical conditions, including but not limited to PVD. The devices can be advantageously configured to achieve a variety of beneficial effects, including but not limited to revascularization and/or angiogenesis via collateralization and/or an increase in microvascular density.

Calcium buildup in peripheral vessels can contribute to the development of PAD. Over time, mineral deposits, such as calcium phosphate deposits, can build up in the intimal or medial parts of blood vessels. These deposits can harden into plaques, which may contribute to such vessels becoming narrower and more rigid, and thus less responsive to normal arterial function, compared to vessels without plaque buildup. In some instances, calcified lesions can begin to form inside a vessel wall and proceed to incorporate the whole vessel wall, eventually becoming diffuse. Hardening and/or narrowing of blood vessels can limit blood flow, which may contribute to development of diseases such as PAD. By providing a TUS wearable device that can focus an acoustic field at or near a region of vascular calcium within a vessel (e.g., calcium deposits, calcified lesions, calcified vessel walls and/or surfaces, and the like) and external of the vessel walls and/or surfaces, the device can be configured to promote revascularization of the blood vessels, such as by increasing vessel elasticity and/or flexibility from cavitation microbubbles interacting with the region of vascular calcium via localized shockwaves (such as further described herein).

Current treatments for plaque-clogged blood vessels include medications and invasive interventions. Patients with asymptomatic PAD can be treated with anti-platelet medications (e.g., aspirin, clopidogrel) and cholesterol-lowering medications such as statins in an attempt to reduce lower extremity atherosclerotic burden. However, approximately 75% of patients progress to develop symptoms of claudication or CLI. Patients with claudication have been shown to benefit from supervised exercised therapy and the medication cilostazol; however, approximately 30% progress with worsening claudication, or to develop CLI. Revascularization with bypass surgery, angioplasty or stent implantation is recommended for refractory claudication or CLI, but is associated with 20-40% rates of restenosis. As such, these advances in catheter-based therapies including angioplasty, stenting, and drug-coated balloons, as well as surgical bypass treatments, have not yet resulted in a high degree of successful treatment for CLI.

Invasive interventions may be expensive, time-consuming, and difficult to implement. Advantageously, by providing an extracorporeal TUS wearable device, such as described herein, PAD interventions can be significantly cheaper than when using devices/procedures requiring breakage of the skin barrier and/or insertion within the lumen of blood vessels, and may be cheaper without reducing (or mitigating reduction of) the efficacy of treatment. For example, extracorporeal treatment via the TUS wearable device may provide improved targeted treatment such as when implemented as and/or used in combination with imaging systems, as described herein. Additionally, extracorporeal treatment can be much simpler than invasive interventions and may reduce (or eliminate) trauma to the body otherwise induced by invasive treatment, and reduce (or eliminate) recovery times. Consequently, the TUS wearable device may be utilized in outpatient settings and increase patient access to much-needed PAD treatment due to both lower costs and lower medical resource/logistical needs.

In some cases, certain invasive surgical interventions may altogether be unavailable to patients because their blood vessels have become too rigid from calcium buildup. For example, due to hardening of the vessel walls, a balloon catheter may not be able to expand the blood vessel sufficient to implant stents. In such instances, more complicated, expensive, and painful interventions may be required, such as bypass surgeries. However, by providing a TUS wearable device that can focus an acoustic field at or near a region of vascular calcium within a vessel and from the outside, the device can be configured to condition the region of vascular calcium to allow subsequent (e.g., secondary) PAD intervention. In some embodiments, the TUS device may be used in combination with secondary PAD interventions to otherwise remove calcium from arterial vessels. For example, the device may be configured to condition the region of vascular calcium via cavitation microbubbles imparting localized shockwaves at the level of the calcium. Conditioning the region of vascular calcium can improve vascular elasticity and/or flexibility, permitting the vessel to expand sufficient for catheter-based revascularization such as stent implantation, balloon angioplasty (e.g., drug-coated balloon angioplasty), and/or other invasive procedures. In some embodiments, the TUS device may be used in combination with minimally invasive secondary PAD treatments, such as shockwave atherectomy, orbital atherectomy, and/or the like, to otherwise remove calcium from arterial vessels. Conditioning vascular calcium can include reducing the hardness and/or rigidity of the vascular calcium (e.g., softening the vascular calcium) such as by disrupting/fracturing (e.g., inducing microfractures in the vascular calcium) via acoustic shockwaves.

Current TUS devices are not designed for the human lower extremity, including the calf in some cases, and their acoustic amplitudes, frequencies, and fields are insufficient to generate the vascular bioeffects necessary for reperfusion of PAD patients. Moreover, such non-invasive devices generate acoustic shockwaves within the lumen of the blood vessels that can cause trauma/damage to the blood vessels. As such, more efficacious systems and methods are needed, including wearable systems that can generate acoustic shockwaves external of the lumen and at the level of the calcium to provide relatively long-term clinical benefits such as increased blood flow and symptomatic relief while reducing (or eliminating) collateral damage from the ultrasound therapy itself. By providing a TUS wearable device that can focus an acoustic field at or near a region of vascular calcium within a vessel and external of the vessel walls and/or surfaces, the wearable device can be configured to generate cavitation microbubbles at or near the region of vascular calcium and external of said walls and/or surfaces. The cavitation microbubbles may impart localized acoustic shockwaves at the level of calcium and lead to profound and unexpected improvements in therapeutic results, including but not limited to increased blood flow from, for example, increased vascular elasticity and/or flexibility. Furthermore, because the device propagates acoustic waves to the vessel walls and/or surfaces from the outside, the reflectivity (e.g., acoustic impedance) of the calcium can limit the ability of the device to generate cavitation microbubbles within the lumen of the blood vessels, thereby promoting safety during treatment.

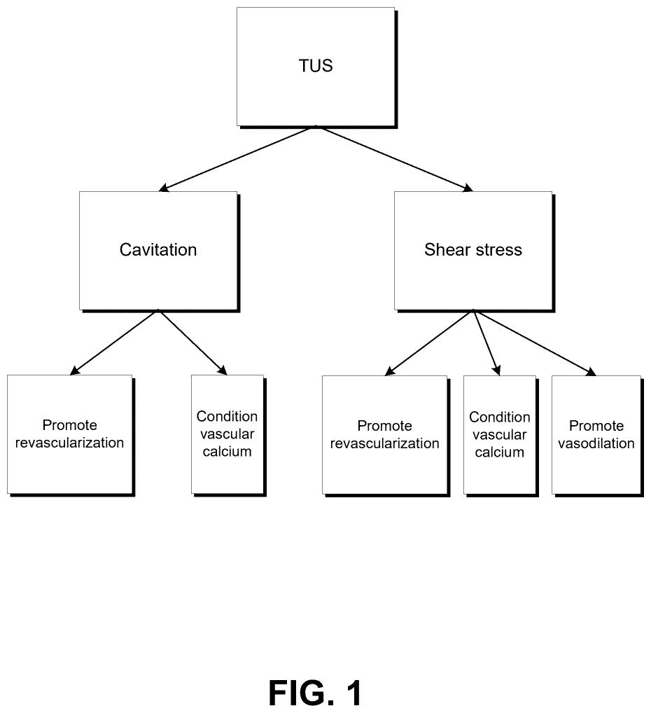

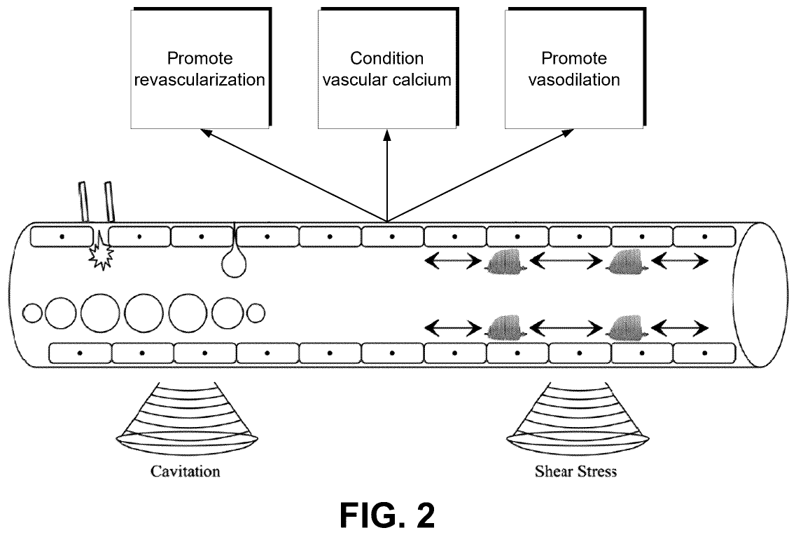

FIGS. 1 and 2 illustrates potential mechanisms of action of, for example, TUS therapy. Not to be limited by theory, such a device can be configured to promote revascularization from cavitation bubbles interacting with a region of vascular calcium at the level of the calcium, such as where the blood vessels are occluded or maximally stenotic. In some aspects, the device can be configured to promote revascularization from shear stresses from ultrasound waves directed to walls and/or surfaces of vessels that are occluded or maximally stenotic. In some aspects, the device may be configured to promote revascularization using a combination of the aforementioned mechanisms. The device can be configured to condition the region of vascular calcium, which can increase the elasticity and/or flexibility of the afflicted blood vessels and promote revascularization of said blood vessels.

In some embodiments, the device may be configured to increase vascular permeability from cavitation microbubbles interacting with the endothelium, and shear stresses from ultrasound waves directly onto endothelial surfaces, which can stimulate the production and/or release of growth factors, angiogenic factors and signaling molecules such as increase tissue vascular endothelial growth factor (VEGF), endothelial nitric oxide synthase (eNOS), basic fibroblast growth factor (bFGF), adenosine triphosphate (ATP), for example, leading to angiogenesis and/or collateralogenesis. Longer duration treatments can advantageously increase the local and possibly also circulating levels of these angiogenic factors among others, leading to collateralogenesis and increased microvascular density in PAD. Promotion of angiogenesis and collateralogenesis via cavitation microbubbles and shear stress is described in greater detail in at least U.S. patent application Ser. No. 18/829,102, filed on Sep. 9, 2024, and entitled “EXTRACORPOREAL THERAPEUTIC ULTRASOUND FOR PROMOTING ANGIOGENESIS,” the entire contents of which are hereby incorporated herein by reference.

In contrast, some conventional systems and methods merely increase nitric oxide within tissue, thus increasing blood flow temporarily via vasodilation. However, these effects and symptomatic relief can be transient in nature and be limited to the duration of the treatment session or a short period thereafter. Not to be limited by theory, achievement of vasodilation without long-term vascular calcium conditioning and/or angiogenic effects in conventional systems can be due to insufficient peak acoustic pressures and/or durations of therapy, among other reasons. As illustrated in FIGS. 1 and 2, additional mechanisms may involve acute vasodilation lasting at least 1 minute and up to 24 hours, to provide acute symptomatic relief prior to angiogenesis, such as for at least about 1, 2, 3, 4, 5, 10, 15, 20, 30, 45, 60 or more minutes, or at least about 2, 3, 4, 6, 8, 12, 16, 18, or 24 hours, or any value or range within or bounded by any of these values or ranges. In some embodiments, vasodilation can be enhanced via microbubbles, for time periods as noted above, for example after a single or multiple energy applications.

Not to be limited by theory, revascularization and/or conditioning of vascular calcium can occur, for example, through two or more ultrasound-mediated mechanisms, as illustrated, for example, in FIGS. 1 and 2. The first mechanism is cavitation: in some embodiments, TUS waves with sufficient peak negative pressure may cause dissolved gas to come out of solution in a region of vascular calcium and/or tissue, and to convert into microbubbles. In response to TUS, these bubbles then volumetrically oscillate and/or burst, generating shockwaves of high intensity (e.g., high pressure) and temperature localized at the level of the vascular calcium, which can disrupt and/or fragment the vascular calcium such that the vascular calcium softens (e.g., reduces in hardness and/or rigidity). The softened vascular calcium can increase vascular elasticity and/or flexibility of the afflicted arterial vessel, which can promote increased blood flow through the vessel and/or permit use of subsequent invasive PAD interventions such as catheter-based interventions. While the process of cavitation is well-described, the inventors are not aware of previous techniques that specifically harness this process to promote revascularization, and particularly revascularization of the lower extremities such as the thigh, calf, and foot. This mechanism may also trigger up-regulation of several molecular mediators of angiogenesis/collateralogenesis as described further in at least U.S. patent application Ser. No. 18/829,102. In some embodiments, p-can be selected to promote cavitation and revascularization without leading to harmful or lethal vascular damage.

A second mechanism is shear stress: in some embodiments, TUS waves of a desired frequency and sufficient amplitude can directly interact with a region of vascular calcium, which may lead to revascularization and/or conditioning of the vascular calcium for subsequent PAD interventions (e.g., catheter-based revascularization). The acoustic waves can induce mechanical vibrations within and/or about the vascular calcium that can disrupt and/or fragment the vascular calcium such that the vascular calcium softens (e.g., reduces in hardness and/or rigidity). The softened vascular calcium can increase vascular elasticity and/or flexibility of the afflicted arterial vessel, which can promote increase blood flow through the vessel and/or permit use of subsequent invasive PAD interventions as described herein. While the effects of shear stress on revascularizations have been described, the inventors are not aware of previous techniques utilizing TUS to specifically increase vascular calcium shear stress, leading to revascularization, and particularly revascularization of the lower extremities. This mechanism may also lead to vasodilation, collateralogenesis and angiogenesis as described further in at least U.S. patent application Ser. No. 18/829,102.

In healthy vessels, vascular smooth muscle cells (VSMCs) maintain the structural integrity and flexibility of the artery walls. However, under certain conditions, the accumulation of lipids, cholesterol, and other substances can lead to plaque formation within the arterial walls. Over time, these plaques can become calcified, making them hard and rigid. The calcified areas of the plaque create stiff regions within the artery, reducing the vessel's ability to expand and contract. Vascular calcification can occur in different layers of the artery. Intimal calcification happens in the inner layer (intima) of the artery, often associated with atherosclerotic plaques. Medial calcification occurs in the middle layer (media) of the artery, often seen in conditions like diabetes or chronic kidney disease. Healthy arteries are elastic and can expand and contract with each heartbeat to help regulate blood pressure and ensure proper blood flow. Calcification reduces this elasticity, making the arteries stiff and less able to accommodate the pulsatile flow of blood. This loss of elasticity leads to increased arterial stiffness, which contributes to higher blood pressure and increased workload on the heart. A vessel afflicted by vascular calcification can have a reduced ability to dilate (expand) in response to increased blood flow demands, which can limit blood supply to tissues, especially during physical exertion, leading to symptoms like claudication (pain in the limbs due to poor circulation) or angina (chest pain due to reduced blood flow to the heart), among other diseases such as PVD, including PAD.

Conditioning regions of vascular calcium on a local level via cavitation and/or shear stress (or combinations thereof) can reduce the hardness and rigidity of calcified structures. Reductions in hardness and rigidity of vascular calcium can improve vascular elasticity and/or flexibility, which can promote revascularization of the afflicted blood vessels and/or permit such vessels to expand sufficient for invasive PAD interventions as described herein. This effect can correlate in some cases with both TUS amplitude (p⁻) and frequency (with greater shear stress at lower frequencies). In some aspects, cavitation can be a threshold-based phenomenon, which occurs at a given p⁻ and increases with greater intensity.

In some embodiments, a wearable ultrasound-based device can be worn and operated for about or at least about 5, 10, 15, 20, 30, 40, 50, or 60 minutes daily, or about or at least about 2, 3, 4, 5, 6, 7, 8, 9, 10, 11, 12, 15, 18, 24, or more hours at a time (or any value or range within or bounded by any of these values or ranges), either cumulatively in multiple treatment sessions, or continuously in some cases. In some embodiments, the device can be worn and operated for between about 10 minutes and about 20 minutes; between about 20 minutes and about 40 minutes; between about 30 minutes and about 60 minutes; between about 1 hour and about 2 hours; between about 2 hours and about 4 hours; or between about 4 hours and about 8 hours per treatment session. However, in some embodiments the device is worn and operated for about, or no more than about 24, 18, 15, 12, 11, 10, 9, 8, 7, 6, 5, 4, 3, or 2 hours at a time. In some embodiments, the device can be worn and operated about or at least about once, twice, three times, or more daily; or once, twice, or three times weekly. In some embodiments, the device can be worn and operated overnight and/or while a patient is sleeping, such as between about 4 hours and about 10 hours, or between about 5 hours and about 9 hours daily or nightly, 5-7 times a week, or during the day while not sleeping.

In some embodiments, the wearable devices allow for convenient dose-response titrations to readily be performed without requiring long treatments to be performed by a medical provider using timeframes such as disclosed above.

In some embodiments, the ultrasound modality could be TUS, SWT, or a dual-mode combination thereof using one or a plurality of ultrasound transducers. In some embodiments, use of TUS (which may include HIFU, LIPUS, or other pulsed or continuous wave acoustic energies) instead of SWT can advantageously allow for titration of one, two, or more acoustic parameters to achieve a desired angiogenic effect as discussed herein. The parameters can include, for example, frequency, pulse repetition frequency (PRF), pulse duration, duty factor, and pressure amplitudes (peak positive and negative pressures; p⁺, p⁻). Additionally, in some cases, TUS can be advantageous as it allows application of multiple sound/pressure waves in each pulse; SWT provides a single pressure wave.

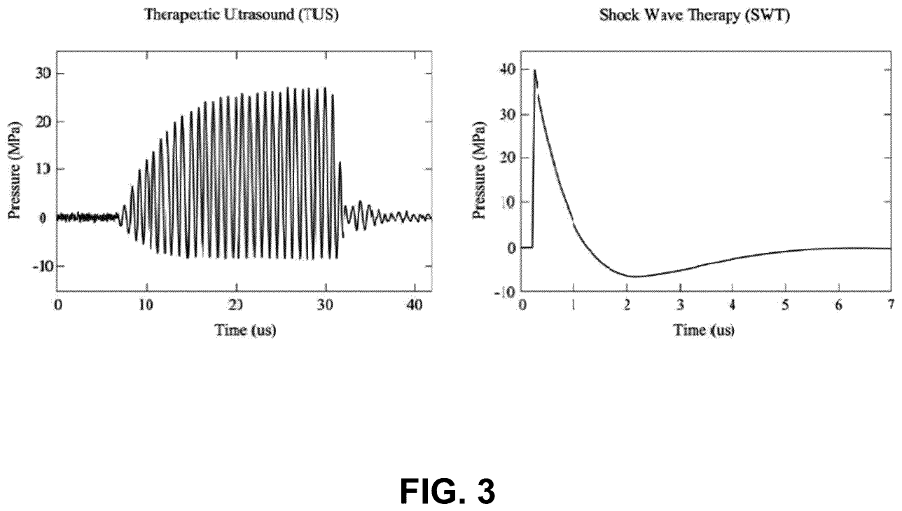

Due to differences in the SWT and TUS waveforms, SWT parameters can only be adjusted to modulate pulse repetition frequency and acoustic amplitudes. FIG. 3 illustrates sample waveforms of TUS and SWT, respectively. In contrast, TUS additionally allows modulation of ultrasound frequency, pulse duration, duty factor, and other parameters. Parameters may be titrated to improve these angiogenic effects (low frequency, high p⁻), while avoiding acoustic intensities that may lead to thermal or cavitation-based damage. However, embodiments can also include SWT, including parameters for pulse repetition frequency and amplitudes as described herein. Furthermore, if regulatory requirements specify a maximal acoustic intensity (p⁻, W or W/cm²) to avoid cavitation-based damage, this parameter can be fixed while others can be adjusted to maximize effect. Finally, adverse effects of ultrasound are most prominent in gas-filled organs such as the lung and gastrointestinal tract in which gas unpredictably reflects and may intensify sound waves. Targeting lower extremity muscle and vasculature, which are generally free of air, can advantageously avoid these effects in some cases. In some embodiments, sinusoidal, square, or other voltage waveforms can be input to the TUS transducers.

The above-described potential mechanisms of TUS-induced cavitation and shear stress can be dependent upon p- and frequency, respectively, although total dose of TUS is also determined by pulse repetition frequency (PRF), duty factor (e.g., % of time that TUS is active), and duration of therapy (e.g., time that patient receives TUS). Each of these TUS parameters has a toxic-therapeutic window, which can advantageously be adjusted for a desired clinical result given its wearable design and titratability of TUS parameters.

Some embodiments of the device and method may be used to immediately or quickly increase perfusion for the treatment of acute limb ischemia, such as about or within about 1 minute, 5 minutes, 10 minutes, 15 minutes, 30 minutes, 60 minutes, 2 hours, 4 hours, 6 hours, 12 hours, 18 hours, or 24 hours after the onset of therapy, as well as have longer-lasting effects as disclosed herein. In some embodiments, systems and methods as disclosed herein can include only TUS and not SWT, only SWT but not TUS, or a combination of both SWT and TUS.

### Example Frequency

In some embodiments, the frequency of ultrasound provided could be between about 250 kHz and about 3 MHz, between about 250 kHz and about 1 MHz, between about 250 kHz and about 500 kHz, between about 1 MHz and about 3 MHz, between about 750 kHz and about 1.25 MHz, between about 500 kHz and about 1 MHz, between about 2 MHz and about 3 MHz, or any value or range within or bounded by any of these values or ranges. Not to be limited by theory, lower frequencies can advantageously increase the shear stress mechanism of action. In some embodiments, lower frequencies could also penetrate more deeply into issue, although frequencies that are too low may penetrate too deeply and reach bone on the opposite end of the desired PAD field. In some embodiments, the treatment frequency could be between about 250 kHz and about 1 MHz on the thigh (deeper field from the skin of the medial thigh to the femur); between about 500 kHz and about 1.25 MHz on the calf (deep field from the skin of the posterior calf to the tibia); or between about 750 kHz and about 1.5 MHz on the ankle (shallow field from the skin of the anterior ankle to the bones), and between about 1 MHz and about 3 MHz on the plantar surface of the foot (even shallower field from skin to the tarsal and metatarsal bones), or between about 500 kHz and about 3 MHZ, between about 1 MHz and about 3 MHz, and/or at least about 500 kHz or 1 MHz in any of the aforementioned locations. In some embodiments, the frequency provided can be about, more than about, or no more than about 200 kHz, 250 kHz, 300 kHz, 350 kHz, 400 kHz, 450 kHz, 500 kHz, 550 kHz, 600 kHz, 650 kHz, 700 kHz, 750 kHz, 800 kHz, 850 kHz, 900 kHz, 950 kHz, 1 MHz, 1.1 MHz, 1.2 MHz, 1.3 MHz, 1.4 MHz, 1.5 MHz, 1.6 MHz, 1.7 MHz, 1.8 MHZ, 1.9 MHz, 2 MHz, 2.1 MHz, 2.2 MHz, 2.3 MHz, 2.4 MHz, 2.5 MHz, 2.6 MHz, 2.7 MHz, 2.8 MHz, 2.9 MHz, 3 MHZ, 3.1 MHZ, 3.2 MHz, 3.3 MHZ, 3.4 MHZ, 3.5 MHz, 4 MHZ, 5 MHZ, 10 MHz, 15 MHz, 20 MHz, 25 MHz, 30 MHZ, 35 MHz, 40 MHz, 45 MHz, 50 MHz, 60 MHz, 70 MHz, 80 MHz, 90 MHz, 100 MHz, or any value or range within or bounded by any of these values or ranges. In some embodiments, systems and methods can provide a plurality of different alternating frequencies during treatment, such as 2, 3, 4, or more different frequencies.

### Example Pulse Repetition Frequency

In some embodiments, the pulse repetition frequency can be between about 0.1 Hz and about 100 Hz, between about 1 Hz and about 3 Hz, between about 0.1 Hz and about 1 Hz, between about 0.5 Hz and about 2 Hz, between about 1 Hz and about 5 Hz, between about 5 Hz and about 10 Hz, between about 10 Hz and about 20 Hz, between about 20 Hz and about 100 Hz, or any value or range within or bounded by any of these values or ranges. Not to be limited by theory, higher PRF can increase total delivered ultrasound energy and angiogenic effect, but may also increase transducer heating. In some cases, a very low PRF may lead to insufficient cavitation and shear stress (and only a short-term vasodilation effect), while very high PRF may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the PRF provided can be about, more than about, or no more than about 0.1 Hz, 0.5 Hz, 1 Hz, 1.5 Hz, 2 Hz, 2.5 Hz, 3 Hz, 3.5 Hz, 4 Hz, 4.5 Hz, 5 Hz, 6 Hz, 7 Hz, 8 Hz, 9 Hz, 10 Hz 12 Hz, 14 Hz, 16 Hz, 18 Hz, 20 Hz, 25 Hz, 30 Hz, 35 Hz, 40 Hz, 45 Hz, 50 Hz, 60 Hz, 70 Hz, 80 Hz, 90 Hz, 100 Hz, 110 Hz, 120 Hz, or any value or range within or bounded by any of these values or ranges. In some embodiments, systems and methods can provide a constant or variable PRF.

### Example Pulse Duration

In some embodiments, the pulse duration can be between about 1 μs (3 oscillations of 3 MHz) and about 100 ms, between about 1 ms and about 10 ms, between about 1 μs and about 100 μs, between about 100 μs and about 500 μs, between about 500 μs and about 1 ms, between about 1 ms and about 5 ms, between about 5 ms and about 20 ms, between about 10 ms and about 50 ms, between about 25 ms and about 100 ms in some embodiments, or overlapping ranges thereof. Not to be limited by theory, longer pulses can increase total delivered ultrasonic energy and likely angiogenic effect, but may also increase transducer heating. In some cases, a very low pulse duration may lead to insufficient cavitation and shear stress (and only a short-term vasodilation effect), while very high pulse durations may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the pulse duration provided can be about, more than about, or no more than about 1 μs, 5 μs, 10 μs, 25 μs, 50 μs, 100 μs, 250 μs, 500 μs, 750 μs, 1 ms, 2 ms, 3 ms, 4 ms, 5 ms, 6 ms, 7 ms, 8 ms, 9 ms, 10 ms, 15 ms, 20 ms, 25 ms, 30 ms, 35 ms, 40 ms, 45 ms, 50 ms, 60 ms, 70 ms, 80 ms, 90 ms, 100 ms, 110 ms, 120 ms, or any value or range within or bounded by any of these values or ranges. In some embodiments, each transducer in the array can have a pulse duration of between about 10 μs and about 1 ms. In some embodiments, systems and methods can provide a constant or variable pulse duration.

### Example Duty Factor

In some embodiments, the duty factor can be between about 0.1% and about 50%, such as between about 0.5% and about 2%, between about 0.1% and about 0.5%, between about 1% and about 5%, between about 2% and about 10%, between about 5% and about 20%, between about 20% and about 50%, or about 1% in some embodiments, or overlapping ranges thereof. Higher duty factor can increase total delivered ultrasonic energy and likely angiogenic effect, but may also increase transducer heating. In some cases, a very low duty factor may lead to insufficient cavitation and shear stress (and only a short-term vasodilation effect), while very high duty factors may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the duty factor provided can be about, more than about, or no more than about 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or any value or range within or bounded by any of these values or ranges.

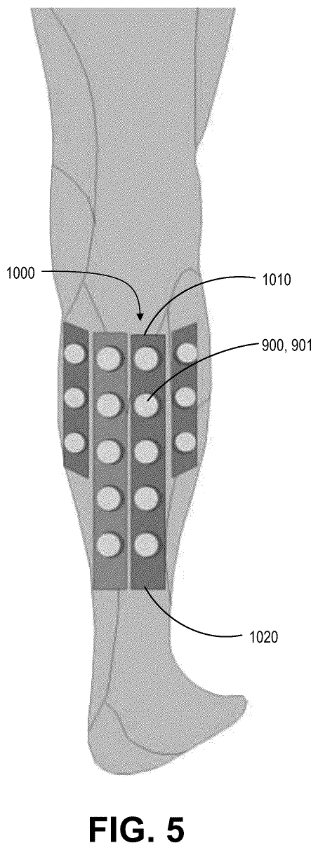

For embodiments incorporating a phased or linear array of transducers, such as illustrated in FIG. 5, each transducer could have a duty cycle that is up to about 1/(total number of transducers). In the example transducer arrangement of FIG. 5, in an array of 5 transducers, each transducer may have up to about a 20% duty cycle. In some aspects, each transducer may have equal duty cycles or unequal duty cycles.

### Example Peak Negative Pressure

In some embodiments, the peak negative pressure (p⁻; greater p- can be associated with more shear stress and cavitation, and revascularization/conditioning effect, although high p-can theoretically lead to vascular damage) can be between about 2 MPa and about 20 MPa, between about 6 MPa and about 10 MPa, between about 2 MPa and about 4 MPa, between about 1.5 MPa and about 4 MPa, between about 1 MPa and about 4 MPa, between about 2.5 MPa and about 3.5 MPa, between about 3 MPa and about 5 MPa, between about 4 MPa and about 6 MPa, between about 5 MPa and about 7 MPa, between about 7 MPa and about 10 MPa or less than about 4 MPa in some embodiments. For clarity, the minus signs preceding the peak negative pressure disclosed herein are omitted—for example, a peak negative pressure of 4 MPa (can be denoted elsewhere as −4 MPa) as described herein is more negative than a peak negative pressure of 1 MPa (can be denoted elsewhere as −1 MPa). In some embodiments, the p⁻ may be selected to maximize sub-lethal cavitation. In some cases, a very low p⁻ may lead to insufficient cavitation and shear stress, while very high p⁻ may lead in some cases to transducer warming, lethal vascular damage (including possible dissection, stenosis, or thromboembolism), microhemorrhage, possible nerve damage, pain, fat or other tissue necrosis, apoptosis, and/or scar formation. In some embodiments, the p provided can be about, more than about, or no more than about 0.5 MPa, 1 MPa, 1.5 MPa, 2 MPa, 2.5 MPa, 3 MPa, 3.5 MPa, 4 MPa, 4.5 MPa, 5 MPa, 5.5 MPa, 6 MPa, 6.5 MPa, 7 MPa, 7.5 MPa, 8 MPa, 8.5 MPa, 9 MPa, 9.5 MPa, 10 MPa, 10.5 MPa, 11 MPa, 12 MPa, 13 MPa, 14 MPa, 15 MPa, 16 MPa, 17 MPa, 18 MPa, 19 MPa, 20 MPa, 21 MPa, 22 MPa, 25 MPa, or any value or range within or bounded by any of these values or ranges.

### Example Acoustic Intensity

In some embodiments, the ultrasound parameters may be configured to provide an acoustic dose as calculated at the surface or the target tissue as described herein to specifically promote revascularization and/or conditioning of a region of vascular calcium. In some embodiments, the acoustic dose as calculated at either the surface, or by derated Iₛₚₜₐ of between about 250 mW/cm² and about 5,000 mW/cm², between about 250 mW/cm² and about 720 mW/cm², between about 720 mW/cm² and about 5000 mW/cm², between about 500 mW/cm² and about 1,000 mW/cm², between about 750 mW/cm² and about 1,500 mW/cm², between about 1 W/cm² and about 2 W/cm², between about 2 W/cm² and about 4 W/cm², between about 3 W/cm² and about 5 W/cm², or overlapping ranges thereof. Derating is a method of making acoustic measurements to account for attenuation in tissue.

In some embodiments, the ultrasound parameters can be configured to provide intensity at the surface, or a derated Iₛₚₜₐ of between about 50 W/cm² and about 1000 W/cm², such as between about 50 W/cm² and about 190 W/cm², between about 190 W/cm² and about 1000 W/cm², between about 150 W/cm² and about 300 W/cm², between about 200 W/cm² and about 500 W/cm², or between about 500 W/cm² and about 1000 W/cm², or overlapping ranges thereof. In some embodiments, the intensity at the surface, or a derated Iₛₚₜₐ provided can be about, more than about, or no more than about 150 mW/cm², 200 mW/cm², 250 mW/cm², 300 mW/cm², 350 mW/cm², 400 mW/cm², 450 mW/cm², 500 mW/cm², 550 mW/cm², 600 mW/cm², 650 mW/cm², 700 mW/cm², 750 mW/cm², 800 mW/cm², 850 mW/cm², 900 mW/cm², 950 mW/cm², 1,000 mW/cm², 1,250 mW/cm², 1,500 mW/cm², 1,750 mW/cm², 2,000 mW/cm², 2,250 mW/cm², 2,500 mW/cm², 2,750 mW/cm², 3,000 mW/cm², 3,250 mW/cm², 3,500 mW/cm², 3,750 mW/cm², 4,000 mW/cm², 4,250 mW/cm², 4,500 mW/cm², 4,750 mW/cm², 5,000 mW/cm², or any value or range within or bounded by any of these values or ranges. In some embodiments, the intensity can be, for example, between about 500 mW/cm² and about 5,000 mW/cm² or between about 1,000 mW/cm² and about 4,000 mW/cm².

In some embodiments, the ultrasound parameters can be configured to provide a mechanical index (MI, defined as MI=p⁻_{d}/√f, where p⁻_{d} is derated peak negative pressure and f is frequency) of between about 1 and about 10, such as no more than about 1.9, between about 2 and about 10, between about 1 and about 4, between about 4 and about 10, between about 1 and about 2, between about 2 and about 4, between about 3 and about 5, between about 4 and about 8, or between about 5 and about 10 in some embodiments, or overlapping ranges thereof. In some embodiments, the mechanical index provided can be about, at least about, or no more than about 0.5, 1, 2, 3, 4, 5, 6, 7, 8, 9, 10, 11, 12, or any value or range within or bounded by any of these values or ranges.

In some embodiments, the system could be configured to deliver ultrasound energy in continuous wave (CW) mode, pulse wave (PW) mode, or both modes.

In some embodiments, the system can be configured to deliver energy with a surface intensity-vessel depth ratio to preferentially treat the target tissue (e.g., region(s) of vascular calcium). The surface intensity-vessel depth ratio can be, for example, about or less than about 0.75 W/cm³, 0.70 W/cm³, 0.65 W/cm³, 0.60 W/cm³, 0.55 W/cm³, 0.50 W/cm³, 0.45 W/cm³, 0.40 W/cm³, 0.35 W/cm³, 0.30 W/cm³, 0.25 W/cm³, 0.20 W/cm³, 0.15 W/cm³, or 0.10 W/cm³ in some embodiments, or any value or range within or bounded by any of these values or ranges, but in some cases at least about 0.05 W/cm³, 0.075 W/cm³, 0.10 W/cm³, 0.125 W/cm³, 0.15 W/cm³, 0.175 W/cm³, or 0.20 W/cm³.

In some embodiments, the surface intensity-vessel depth ratio is between about 0.10 W/cm³ and about 0.60 W/cm³, between about 0.10 W/cm³ and about 0.55 W/cm³, between about 0.125 W/cm³ and about 0.50 W/cm³, or between about 0.20 W/cm³ and about 0.50 W/cm³. Not to be limited by theory, such ratios among others have unexpectedly been found to advantageously treat PAD and other indications as described herein in some cases by focused ultrasound delivery to the target tissue while minimizing off-target effects.

In some embodiments, the intensity to surface area of the skin overlying the target tissue (e.g., vessels having region(s) of vascular calcium) can be about, less than about, or at least about 5:1, 4.5:1, 4:1, 3.5:1, 3:1, 2.5:1, 2:1, 1.5:1, 1:1 or less or any value or range within or bounded by any of these values or ranges. Such ratios have unexpectedly been found to advantageously treat PAD and other indications as described herein in some cases.

In some embodiments, the maximum pulse power delivered can be between about 30 W and about 70 W, between about 40 W and about 60 W to foot vessels (e.g., foot vessels having region(s) of vascular calcium), or about or no more than about 75 W, 70 W, 65 W, 60 W, 55 W, 50 W, 45 W, 40 W, 35 W, 30 W, 25 W, or any value or range within or bounded by any of these values or ranges. In some embodiments, for calf vessels (e.g., calf vessels having region(s) of vascular calcium), the maximum power delivered can be, for example, between about 100 W and about 250 W, between about 125 W and about 225 W, or about or less than about 250 W, 245 W, 240 W, 235 W, 230 W, 225 W, 220 W, 215 W, 210 W, 205 W, 200 W, 195 W, 190 W, 185 W, 180 W, 175 W, 170 W, 165 W, 160 W, 155 W, 150 W, 145 W, 140 W, 135 W, 130 W, 125 W, 120 W, 115 W, 110 W, 105 W, 100 W, or less, or any value or range within or bounded by any of these values or ranges.

In some embodiments, pulse power delivered by a transducer (such as any of the transducers described herein) operating at, for example, a p of about 3.4 MPa and having a pulse duration of about 1 ms can have a pulse power between about 250 W and about 600 W, such as between about 340 W and about 500 W and/or any overlapping ranges thereof. For example, such a TUS transducer may have a max pulse power of 250 W, 260 W, 270 W, 280 W, 290 W, 300 W, 310 W, 320 W, 330 W, 340 W, 350 W, 360 W, 370 W, 380 W, 390 W, 400 W, 410 W, 420 W, 430 W, 440 W, 450 W, 460 W, 470 W, 480 W, 490 W, 500 W, 510 W, 520 W, 530 W, 540 W, 550 W, 560 W, 570 W, 580 W, 590 W, 600 W, any value therebetween, or any range incorporating any two of the aforementioned values. A TUS transducer may deliver any level of power within the aforementioned ranges such as according to multiple different TUS parameters, including but not limited to, drive frequency and/or other transducer-specific characteristics. Power values according to different p-values (e.g., drive pressures) may be calculated based on a pulse power adjustment factor, such as according to the following equation:

$$PPAF = {( \frac{{p}_{i}^{-}}{{p}_{0}^{-}} )}^{2} ,$$

wherein PPAF is the pulse power adjustment factor, p⁻ᵢ is the new peak negative pressure, and p⁻₀ is the original peak negative pressure. For example, to determine a maximum pulse power delivered by a transducer having a pulse duration of about 1 ms and operating at a p of about 1.7 MPa instead of about 3.4 MPa, the PPAF would be

$${( \frac{1.7 MPa}{3.4 MPa} )}^{2} = 0.25 .$$

Thus, the maximum pulse power delivered by the transducer would be about 0.25*600 W=150 W. Various pulse powers may be calculated accordingly.

In some embodiments, the surface power-intensity ratio of the ultrasonic energy delivered can be about, at least about, or no more than about 1 cm², 2 cm², 3 cm², 4 cm², 5 cm², 6 cm², 7 cm², 8 cm², 9 cm², 10 cm², 11 cm², 12 cm², 13 cm², 14 cm², 15 cm², 16 cm², 17 cm², 18 cm², 19 cm², 20 cm², 21 cm², 22 cm², 23 cm², 24 cm², or 25 cm², or any value or range within or bounded by any of these values or ranges and selected to better focus ultrasound to the target tissue. In some embodiments, the surface power-intensity ratio can be, for example, between about 3 cm² and about 25 cm², between about 3 cm² and about 5 cm², between about 15 cm² and about 25 cm², or less than about 25 cm², 20 cm², 15 cm², 10 cm², 5 cm², or less.

In some embodiments, the therapeutic energy can be focused to a particular depth depending on the desired target tissue, e.g., region of vascular calcium within a vessel. In some aspects, the therapeutic energy can be focused on any arterial region within the body of a patient/subject, such as any region of the main arteries. In some aspects, the therapeutic energy can be focused on arterial regions of the lower extremities, such as arterial regions in the thigh, calf, and foot. Such arteries include the iliac artery, the common femoral artery, the superficial femoral artery, the popliteal artery, the anterior tibial artery, the posterior tibial artery, subclavian artery, axillary artery, brachial artery, radial artery, ulnar artery, renal arteries, carotid arteries, celiac trunk/artery, superior mesenteric artery, and inferior mesenteric artery, among others. In some aspects, the therapeutic energy can be focused on arterial regions in the upper extremities of the body, such as arterial regions of the torso, arms, hands, and neck. Any number of the aforementioned arterial regions can be treated to create a therapeutic effect (e.g., increased blood flow, such as via revascularization and/or conditioning of regions of vascular calcium) using system and methods described herein.

In some embodiments, for regions of vascular calcium in the calf, the energy can be focused to a vessel depth of, for example, between about 2 cm and about 8 cm, between about 3 cm and about 9 cm, such as between about 4 cm and about 8 cm, or between about 4.5 cm and about 7 cm. In some embodiments, for regions of vascular calcium in the foot, the energy can be focused to a vessel depth of, for example, between about 1 cm and about 4 cm, such as between about 1.5 cm and about 3.5 cm, or between about 2 cm and about 3 cm. In some embodiments, the energy can be focused to a vessel depth of about, at least about, or no more than about 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, 10 cm, 11 cm, 12 cm, 15 cm, or any value or range within or bounded by any of these values or ranges. In some embodiments, energy can be directed to one or more of the anterior, lateral, medial, and/or posterior calf/lower leg to treat an arterial vessel and/or associated muscles, such as those disclosed herein.

As some non-limiting examples, in some embodiments, delivering TUS ultrasonic energy to a calf or foot arterial region at a frequency of between about 1 MHz and about 3 MHz, a peak negative pressure of between about 2 MPa and about 4 MPa, energy delivery of between about 1 W/cm² and about 4 W/cm² at the target tissue level, and a surface intensity-vessel depth ratio between about 0.10 W/cm³ and about 0.60 W/cm³, for a cumulative total of about or at least about 10 minutes, 20 minutes, 30 minutes, 40 minutes, 50 minutes, 60 minutes, or more cumulative minutes per week for at least about 1, 2, 3, 4, 5, 6, 7, 8, 9, 10, or more weeks can unexpectedly promote revascularization in some cases.

### Example Device

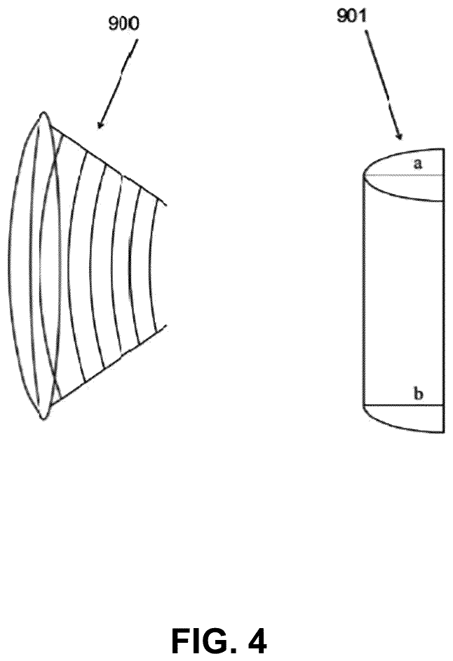

FIG. 4 illustrates an embodiment of an example single-element transducer 900 having a circular geometry with a spherical curve (on the left), as well as an example transducer 901 having a rectangular geometry with a cylindrical curve (on the right). In some embodiments, the transducer 900, 901 can include a taper with multiple radii of curvature including first radii of curvature (a) and second radii of curvatures (b) as illustrated in the right-hand embodiment.

In the example of FIG. 4, the discussion of embodiments of spherical and cylindrically focused transducers that are concave, and thus conform to the curvature of the lower extremity, is not intended to be limiting. In some embodiments, transducers 900, 901 may be convex, in which the acoustic field becomes wider than the cross-sectional area of the transducer. Such a transducer may require higher power to achieve equivalent acoustic pressures at depth, but can allow for treatment of a larger area of tissue with a transducer of equivalent cross-sectional area.

In some aspects, non-convex designs including multiple transducers oriented at varying radial directions of focus within a flat array of transducers may also achieve a divergent or spreading ultrasound beam for maximum muscle and vascular target coverage with minimal transducer size.

In some embodiments, the transducer 900, 901 can be configured to conform to the curvature of lower extremities for optimal therapeutic effect and wearability. For example, both the transducer 900, 901 and battery/generator that supplies power to the transducer 900, 901 may be gently curved to conform to the curvature of the thigh, calf, ankle, or foot. Fixed curvature transducers may be fabricated based on average human lower extremity curvatures. The curvature could be, for example, spherical or cylindrical, and have a single or multiple radii of curvature along the length to best conform to a patient's/subject's anatomy. This can advantageously minimize device bulk, increase comfort, limit the volume of coupling material (e.g., adhesive, coupling gel, gel pack, etc.), and reduce (or minimize) risk of air incorporation into the transducer-tissue interface. In some embodiments, the system can include a single device incorporating a removable/rechargeable power supply and one or more transducer arrays. In some embodiments, the device can include a sleeve that can conform to any region of the lower extremities, such as the wearable sleeve described further in at least U.S. patent application Ser. No. 18/829,102. In some embodiments, a single device for the entire lower extremity can treat all of the arterial regions from thigh to foot, thigh to calf, or calf to foot.

Transducers 900, 901 can have a diameter of between about 5 mm and about 30 mm, such as about 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, or any value or range within or bounded by any of these values or ranges. In some embodiments, the transducer diameters are between about 20 mm and about 30 mm, such as about 25 mm in some cases.

In some embodiments, a transducer 900, 901 configured to be placed on a thigh of a patient can have a radius of between about 10 cm and about 30 cm, and have a radius of curvature of between about 50 cm and about 300 cm. A transducer 900, 901 configured to be placed on a calf of a patient can be configured to have a radius of between about 5 cm and about 15 cm, and a radius of curvature of between about 25 cm and about 150 cm. A transducer 900, 901 configured to be placed on an ankle or plantar midfoot of a patient can have a radius of between about 2.5 cm and about 10 cm, and a radius of curvature of between about 10 cm and about 100 cm. In some embodiments, transducers 900, 901 could have one or more of: a radius of curvature of between about 5 cm and about 100 cm; thickness of between about 100 μm and about 1 cm; composite, sputter coated, or fiber-based ultrasound transducers conformable to the skin surface.

In some embodiments, flexible piezoelectric materials may be used to allow alteration of transducer shape to fit a patient's lower extremity segment. Flexible and stretchable electronic materials exist which may contain piezoelectric materials and may be much thinner than conventional piezoelectric materials. Some examples of composite piezoelectric materials can take the form of pastes or paints in the form of soft or malleable materials. These materials are also referred to as piezoelectric thick film materials or piezoelectric paint. Such materials can be integrated, for example, in a band-aid design and thus adhered to the patient's anatomy.

Transducers 900, 901 may include solid piezoelectric materials (PZT) with various backing materials (air, epoxy, and/or glass microbeads). Some embodiments may use thick or thin piezoelectric ceramic or polymer films that may be integrated using flexible transducer surfaces to allow for conformal or flexible apposition to the body. Lead zirconate titanate (PZT), lead-free piezoelectric thin films, piezopolymer films, cellulose-based electroactive paper, and other materials may be used. In some embodiments, the transducer material can include any of the following: PZT-4 simple composite, PZT-4 bulk ceramic, PZT-4 Air-kerf composite, and/or PZT-5A composite. Films may be deposited before device fabrication, and potential advantages for application in PAD treatment include lower weight and cost, lower power requirements, wide frequency range of operation, and large amplitudes with lower driving voltages and hysteresis.

In some embodiments, the transducers 900, 901 can be bulk (e.g., bulk ceramic) and/or composite (e.g., composite ceramic fiber) transducers. Advantageously, composite transducers can exhibit purer resonance (from thickness mode alone instead of lateral) compared to that of bulk transducers. Conversely and advantageously, bulk transducers may be less expensive than composite transducers (with lower cost disparity with larger transducer diameter) and exhibit less damping compared to that of composite transducers (although damping exhibited by composite transducers may be reduced with purer ceramic composites). Based on water bath testing, bulk designs may also have near field acoustic intensity hot spots that may cause cavitation within 1-2 cm of the skin surface, which can result in adverse effects including burning and/or irritation. These near field effects can be minimized by increasing transducer diameter and thus amplifying thickness mode over lateral resonances, or by utilizing composite transducers (which may not exhibit acoustic intensity “hot spot” effects).

In some embodiments, the transducers 900, 901 are housed within a polymer housing, such as a thermoplastic such as PEEK, and covered in a polymer layer (e.g., polyimide) that opposes the skin. The polymer housing can be configured to minimize weight of each transducer and minimize heat conduction to the skin.

FIG. 5 illustrates an example embodiment of a plurality of transducers arranged in an array 1000 such as a linear array. The transducer array 1000 can include any of the transducers described herein, such as transducers 900, 901 described with reference to FIG. 4. In some embodiments, one or more transducer arrays 1000 can be used to deliver TUS to an arterial region. In the example of FIG. 5, one or more linear arrays 1000 of transducers 900, 901 are positioned on the posterior of the calf of a patient to deliver TUS to arterial regions (e.g., regions of vascular calcium) of the calf; however, this is not intended to be limiting. One or more arrays 1000 can be secured to other portions of the lower extremities, such as the thigh, ankle, or foot to treat associated arterial regions. In some embodiments, one or more arrays 1000 may be secured to one or more portions of the upper extremities, such as the torso, arms, hands, or neck to treat associated arterial regions. In some embodiments, other kinds of arrays, such as phased arrays, may be used to deliver TUS to regions of vascular calcium. In some embodiments, combinations of various different kinds of arrays (e.g., linear phased array) may be used to deliver TUS to regions of vascular calcium.

The array 1000 can include a proximal end 1010 (e.g., top end) and a distal end 1020 (e.g., bottom end). In the example of FIG. 5, proximal ends of two or more arrays 1000 may be horizontally aligned. In some embodiments, distal ends of two or more arrays 1000 may be horizontally aligned. In some embodiments, proximal and/or distal ends of two or more arrays 1000 may not be horizontally aligned. Similarly, proximal and/or distal ends of two or more arrays 1000 may be vertically aligned. In some embodiments, arrays 1000 may be offset proximally with respect to the midpoint of the calf (e.g., the midpoint of the posterior side of the calf). Lateral arrays, in some embodiments, may be offset proximally with respect to the midpoint of the medial arrays. Arrays 1000 may be centered or offset distally.

An array 1000 may contain multiple transducers 900, 901 (e.g., 2, 4, 6, 8, 10, 12, 16, 24, 32, 64, 128, 256, 512 or more, (or any value or range within or bounded by any of these values or ranges) operating with none, one, some, or all transducers out of phase with each other. For example, the transducer array 1000 may be a linear array in which all transducer elements 900, 901 operate in phase with one another. In some examples, the transducer array 1000 may be a phased array in which one or more transducer elements 900, 901 operate out of phase with one another, such as about 90, 180, or 270 degrees out of phase with one another. Transducer elements 900, 901, in some embodiments, may be geometrically focused and/or electronically focused, as further described herein. In some embodiments, the array 1000 of transducer elements can be configured for mechanical and/or electronic beam steering of a generated acoustic field, as further described herein.

Each transducer element 900, 901 in an array 1000 can be spaced sufficiently apart so as to avoid acoustic interaction of side lobe artifacts emanating from one transducer with adjacent transducers. The width of side lobes may be measured prior to array fabrication, and elements can thus be spaced accordingly.

In some embodiments, array elements 900, 901 can conform to any desired anatomical shape for more efficient and complete energy delivery to a target region. Transducer size may be selected based on a specific target (e.g., muscle area) for greater tissue coverage. Each element (e.g., transducer) 900, 901 of the array 1000 may be flat, circular, oval, spherical, cylindrical, rectangular, or any other shape. The surface area of each transducer element 900, 901 may be, in some embodiments, between about 2 cm² and about 100 cm². Circular elements, for example, may have diameters between about 1 cm and about 20 cm, such as about 4 cm, about 6 cm, or about 9 cm in some embodiments. The individual array elements 900, 901 may be arranged within a transducer housing having non-limiting shapes (such as rhombus, oval, trapezoidal) to specifically conform to the anatomic treatment area. In some embodiments, individual transducer elements 900, 901 can be fabricated with multiple different curvatures and sizes to account for anatomic variations. As described herein, in some embodiments, polygons with short axes substantially parallel to the direction of curvature can be used to advantageously allow for conformability/flexibility while maintaining maximal surface area coverage.

The transducer array 1000 can be conformable to the anatomical target region for more efficient and complete energy delivery. For example, the transducer array 1000 can include flexible materials and generally conform to the shape of any anatomical region of the lower and/or upper extremities of a patient. In some examples, a flexible substrate can be used to create a conformable array 1000. Polyimide, as one example of a polymer, may be used to laminate the transducer array 1000 together to create conformity. The polymer may also be reinforced with a substrate, e.g., alloy braids or a textile.

In some embodiments, the array-based device can allow individual array elements/transducers to be temporarily inactivated by the user or the system if they are outside the desired anatomic treatment area. The remaining elements that are placed properly over the anatomic therapeutic area can still provide the desired energy delivery. In some embodiments, transducers 900, 901 can include an array of multiple, flexible, thin composite ultrasound transducers that are each conformable.

The array 1000, in some embodiments, may be shaped to target a longitudinal section of a target vessel. For example, the array 1000 may be secured to a patient such that it extends along a skin surface overlaying a longitudinal section of the target vessel. In some examples, the array 1000 may have a long axis that extends along a length of up to about 20 cm or more. The array 1000 may have a long axis that extends along a length of less than about 20 cm. In some examples, the long axis of the array 1000 may have a length that corresponds to the longitudinal length of a portion of an arterial vessel such as a region of vascular calcium. The short axis of the array 1000, in some examples, may have a width that corresponds to the width of the target vessel (or is wider or narrower than the width of the target vessel). In some embodiments, multiple arrays 1000 may be used to target a longitudinal section of a target vessel. For example, arrays 1000 may be positioned proximate each other (e.g., side by side, end to end) to target a larger portion of a target vessel than can be targeted by any one array alone.

An array of transducer elements may be secured to a patient via any number of mechanical and/or chemical means, including but not limited to, a sleeve, straps, bands, clips, clamps, hook-and-loop fasteners, suction, adhesives (e.g., gels, skin-friendly glue, tape, cream, etc.), and the like. A transducer surface can be coated with a material to prevent corrosion from frequent exposure to ultrasound gel and/or adhesive material. In some embodiments, the array 1000 may be incorporated into a sleeve and/or secured to the patient via a sleeve such as further described in at least U.S. patent application Ser. No. 18/829,102.

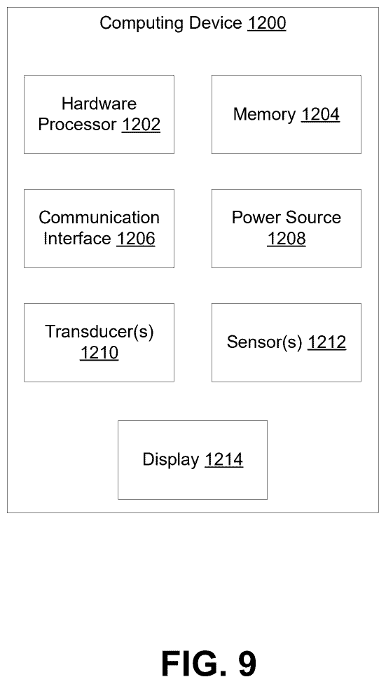

In some embodiments, each transducer element 900, 901 of the array 1000 can be in electrical communication (e.g., wired) with a battery/generator/interface console to allow programming for pulsed or continuous wave TUS. In some aspects, the pulse duration of acoustic waves may be varied (e.g., by a user, by the system) based on, for example, the needs of the patient. For example, a vessel having more vascular calcium may require greater amounts of TUS (e.g., more cavitation bubbles of greater intensity) than does a vessel having less vascular calcium. In some examples, long pulse durations may generate a greater number of cavitation microbubbles in the region of the vascular calcium than can short pulse durations. As further described herein with reference to FIG. 9, the wearable device/system can include a portable, removable, and/or rechargeable integrated power source and controller that can connect directly to the ultrasound transducers. The integrated power source and controller can allow a user and/or healthcare provider to specify treatment parameters including one or more of: frequency, treatment time, duty cycle, and/or acoustic intensity.

The transducer array 1000, in some embodiments, may be configured to geometrically and/or electronically focus TUS waves on a region of vascular calcium. Focusing TUS on a region of vascular calcium may include causing emitted acoustic waves to constructively interfere and/or destructively interfere at a certain region of vascular calcium to, for example, increase the intensity of TUS. Focusing TUS may include focusing the TUS at various different focal depths to target various different regions of vascular calcium. For example, transducer elements 900, 901 can be configured to focus the acoustic field approximately between about 2 cm and about 8 cm away from the skin surface, or other distances as disclosed herein.

In some embodiments, the transducer elements 900, 901 of the array 1000 may be arranged in a curvilinear or parabolic shape to permit geometric focusing. Such an arrangement may cause acoustic waves emitted by the transducer elements 900, 901 to converge (e.g., focus) at a specific point, such as the region of vascular calcium. For example, an array 1000 having a parabolic arrangement of transducer elements 900, 901 can focus therapeutic energy at a focus of the parabola.

In some embodiments, the transducer array 1000 may be configured to electronically focus TUS waves on a region of vascular calcium. For example, the phases of signals sent to individual transducer elements 900, 901 may be adjusted to create a virtual curvature in the emitted acoustic wavefront (e.g., based on constructive and/or destructive interference of acoustic waves) such that the TUS can be focused to different regions of the target vessel. In some examples, the system may introduce time delays in sending signals to various transducer elements 900, 901 of the array 1000 to produce electronic focusing similar to that described with reference to phase adjustments.

The example transducer array 1000 of FIG. 5, in some embodiments, may be configured to permit mechanical and/or electrical beam steering. Beam steering can include directing the main lobe of the emitted acoustic field in a specific direction. In some examples, beam steering may be achieved by moving the array 1000 itself or while keeping the array 1000 stationary. In some embodiments, mechanical beam steering can include moving the array 1000 along a portion of a patient's/subject's body part, such as along a skin surface of the patient's/subject thigh, calf, or foot. Movement can occur daily or weekly as desired for maximal therapeutic effect. Vessels in the lower extremities such as the thigh, calf, and foot, extend vertically (or substantially vertically) from the upper leg to the foot. Consequently, a transducer array 1000 may move vertically along a skin surface of the lower extremities to target different regions of vascular calcium of an underlying arterial vessel (e.g., scanning the target vessel). Such movement may occur in an automated manner with an algorithm and motorized translation. For example, the transducer array 1000 may be connected to (or integrated into) a system that facilitates the mechanical translation of the array 1000 along the patient's lower extremities, such as a device (e.g., track-based device or guide rail-based device) that can move the array 1000 vertically along a skin surface of the patient's thigh, calf, ankle, or foot. In some examples, automated translation of the array 1000 may cause the array 1000 to move from a first lower extremity (e.g., a thigh) to a second lower extremity (e.g., a calf). In some examples, automated translation may cause the array 1000 to move along a path on the skin surface that is aligned (or substantially aligned) with the underlying vessel.

In some embodiments, within a single anatomic segment (e.g., thigh, calf, ankle, foot, etc.), the transducer array 1000 may be rotated to target the anterior, posterior, medial, or lateral side of the extremity based on desired target. Such movement may occur in an automated manner with an algorithm and motorized rotation. For example, the array 1000 may be connected to (or integrated into) a system that facilitates the mechanical rotation of the array 1000 about the patient's lower extremities to target anterior, posterior, medial, or lateral sides of an anatomical region, such as a device that can move the array 1000 horizontally/laterally along a skin surface of the patient's thigh, calf, ankle, or foot.

In some embodiments, the transducer array 1000 may be secured, unsecured, and resecured to different portions of the patient's lower (or upper) extremities to target different regions of vascular calcium of an afflicted vessel and/or to target different sides of an anatomical region.

In some embodiments, individual transducer elements 900, 901 may be configured to move while the array 1000 itself remains stationary. For example, a transducer 900, 901 can be connected to an actuator (e.g., a linear actuator) or a tilting mechanism configured to adjust the position of the transducer 900, 901 to adjust the direction of the emitted acoustic waves.

A transducer array 1000, in some embodiments, may be configured to utilize electronic beam steering. In some examples, the system may utilize electric switching and “grab” (e.g., cause one or more transducers to emit acoustic waves and/or prohibit one or more transducers from emitting acoustic waves) different transducer elements (such as a subset of the transducer elements) to emit acoustic waves directed to different portions of a target vessel. For example, the system may activate (e.g., turn on, cause to emit acoustic waves) a first subset of transducer elements 900, 901 disposed at a first portion of a skin surface. Acoustic waves emitted by the first subset of transducers may be directed to a first region of vascular calcium underlying the corresponding first portion of skin surface. After the system treats the first region of vascular calcium, the system may activate a second subset of transducer elements 900, 901 disposed at a second portion of the skin surface. Acoustic waves emitted by the second subset of transducers may be directed to a second region of vascular calcium underlying the corresponding second portion of skin area. For example, the system may cause the second subset of transducers to emit acoustic waves and prohibit the first subset of transducers from emitting acoustic waves. The first and second subsets of transducers may include entirely different transducer elements of the array 1000 or may include one or more of the same transducer elements of the array 1000. By “grabbing” (e.g., selecting) different subsets of transducers 900, 901 disposed at different portions of the array 1000, and by positioning the array 1000 such that it extends along a skin surface overlying an afflicted vessel (e.g., extends vertical or substantially vertically along a skin surface), the system can effectively walk the acoustic field along the length of the transducer array 1000 to target various different regions of vascular calcium of the underlying afflicted vessel.

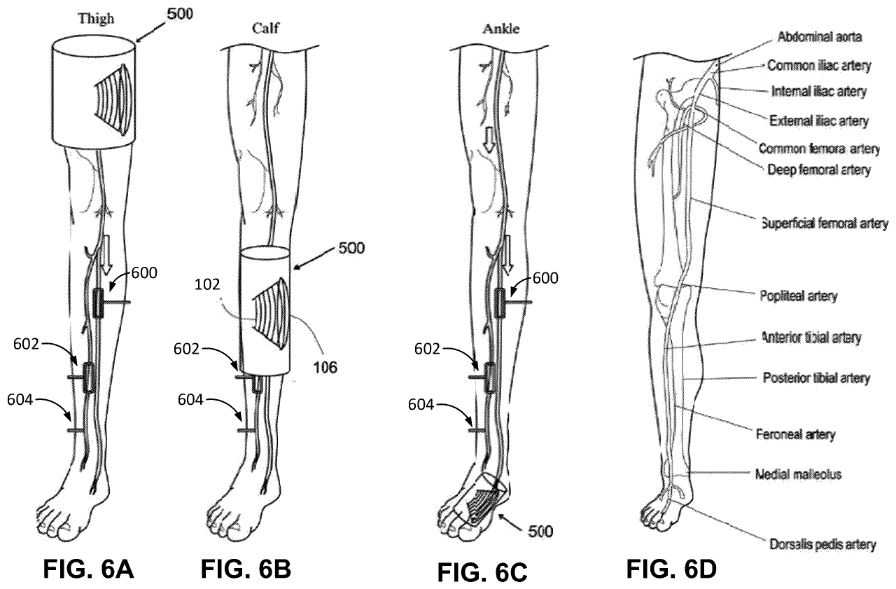

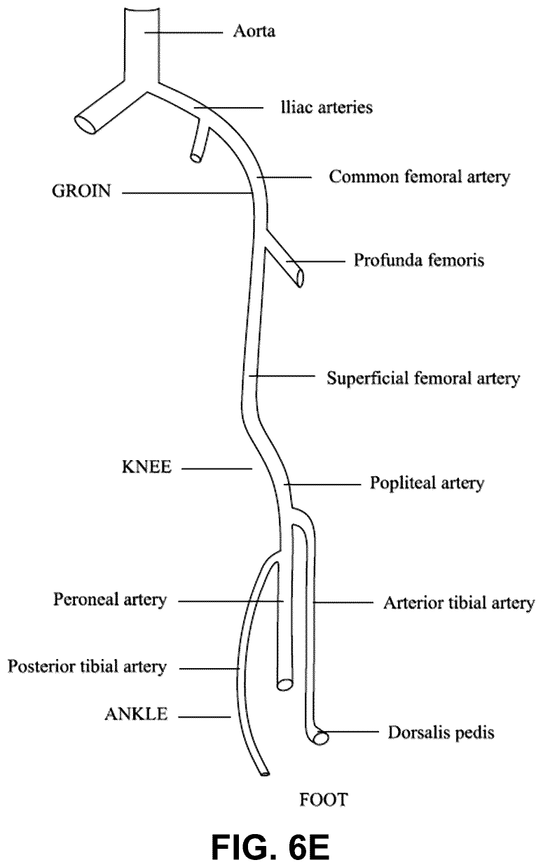

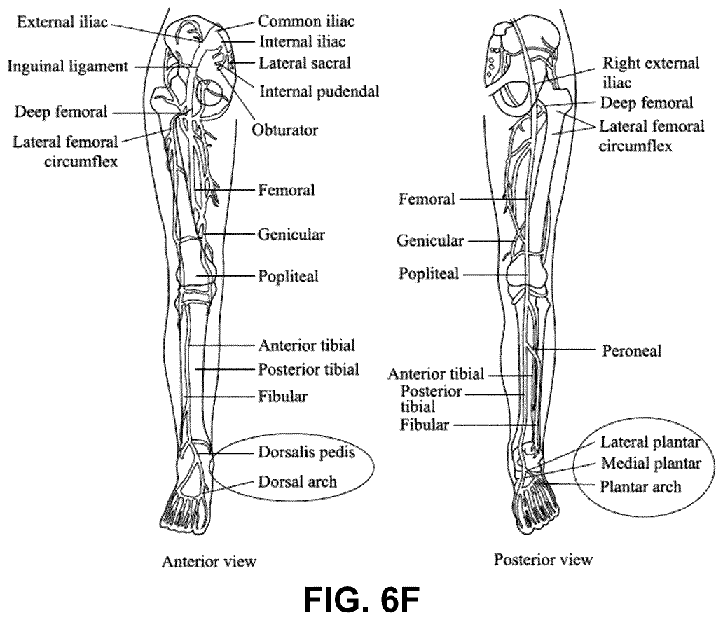

FIGS. 6A-6C illustrate an example embodiment of a TUS wearable device 500 including sleeve 102 and transducer(s) 106 for placement over various different skin surfaces of the lower extremities (e.g., thigh, calf, ankle, foot) for promoting revascularization of any blood vessel of the lower extremities. For purposes of illustration, positions 600, 602, and 604 in FIGS. 6A-6C are marked with a rectangle and corresponding lead line to indicate example treatment locations for application of TUS via wearable device 500; however, such treatment locations are not intended to be limiting. The wearable device 500 may be positioned to target any portion of an arterial vessel, such as any region of vascular calcium. In some aspects, the wearable device 500 may be placed over various different skin surfaces of the lower extremities to condition regions of vascular calcium of underlying arterial vessels to permit subsequent PAD intervention, such as catheter-based revascularization. The transducer(s) 106 and position of the wearable device 500 may be selected based on the region of vascular calcium to be targeted for TUS. For example, positioning of the wearable device 500 at the thigh at a skin surface overlying the femoral artery can promote revascularization of the femoral artery. In some examples, positioning the wearable device 500 at the anterior and/or posterior sides of the calf at a skin surface overlying the anterior and/or posterior tibial arteries can promote vascularization of the anterior and/or posterior tibial arteries. In some examples, positing the wearable device 500 at the foot at a skin surface overlying the dorsalis pedis artery can promote revascularization of the dorsalis pedis artery.

In some embodiments, the wearable device 500 can be implemented as the transducer array 1000 and/or include one or more features and/or functions of array 1000, as described with reference to FIG. 5. In some embodiments, the wearable device 500 can include one or more transducer arrays 1000. Transducer(s) 106 may be implemented as transducers 900, 901 and/or include one or more features and/or functions of transducers 900, 901, as described with reference to FIG. 4.

The device 500 and/or transducer(s) 106, in some embodiments, can be configured to be movable. For example, in some embodiments, the wearable device 500 can be configured to be movable in the direction of the arrows depicted in FIGS. 6A-6C (e.g., translation in a vertical direction). Although the examples of FIGS. 6A-6C illustrate potential stop positions, these are not intended to be limiting. The wearable device 500 may move to and stop (or be moved to and stopped) at any skin surface of the lower extremities. In some embodiments, the wearable device 500 can be configured to be movable within a single anatomic segment (e.g., thigh, calf, ankle, foot, etc.) to target anterior, posterior, medial, and/or lateral sides of the extremity, such as rotatable about the anatomic segment.

Although the examples of FIGS. 6A-6C illustrate a wearable device in the form of a sleeve configured to conform to the shape of any anatomic region, this is not intended to be limiting. The TUS wearable device 500 can be implemented as or include one or more transducer arrays (e.g., array 1000) secured to the patient/subject via various different mechanical and/or chemical means, such as described herein.

FIGS. 6D-6F illustrate various different non-limiting arterial vessels that may be involved in PAD and treated using systems and methods described herein (along with other anatomical landmarks). In some embodiments, systems and methods can be configured for vessel-specific positioning of the wearable device 500 to position major vessels and/or muscles in the near-field and bone in the far-field. In some aspects, the wearable device 500 can include specific anatomic markers to guide placement (e.g., about the 3 o'clock position on the medial thigh; 6 o'clock on the posterior calf, and 12 o'clock position on the plantar mid-foot wherein 12 o'clock is generally the anterior surface of the leg and foot (e.g., the patient's shin)).

In some embodiments, a larger wearable device 500 (e.g., a longer transducer array 1000) may be developed to target two or more lower extremity segments, including a separate single-element transducer or separate array for each of the segments.

Depending on transducer size and design, the entire segment of thigh, calf, ankle, or plantar foot may or may not be able to be treated with a transducer in one location. Furthermore, maintaining a transducer at the same skin location may predispose to infection, contact dermatitis, or simply discomfort. The wearable device 500 may be repositioned longitudinally or horizontally along the lower extremity segment, or rotatably about the lower extremity segment, to reduce (or eliminate) such effects.

It is advantageous that the ultrasound delivered by the wearable device 500 be well-tolerated by the patient. However, at certain parameters, it is possible that TUS may cause pain, discomfort, or nerve stimulation. Some TUS parameters that could result in these symptoms are p/MI and pulse duration/duty cycle, among others. In some embodiments, these parameters can be controlled to stay below, such as just below, the pain/nerve stimulation threshold and improve tolerance and compliance, and increase clinical effects. The parameters in some embodiments can be adjusted manually by a healthcare provider and/or the patient, such as via a user control on the device or a wired or wireless remote, such as a tablet or smartphone, for example, as further described in FIG. 9.

Acoustic waves may be distorted by air in the interface between the transducer and the skin, reducing delivery of TUS energy to tissue, and/or resulting in transducer heating (due to reflection back to the transducer). As such, stable air-free contact between the transducer and the skin can be advantageous. In some embodiments, ultrasound gel and/or specifically designed ultrasound gel pads may be incorporated between the skin surface and the transducer such that air in the skin-transducer interface is eliminated (or minimized or reduced). For example, as described herein, a transducer and/or the skin surface may be coated in ultrasound gel.

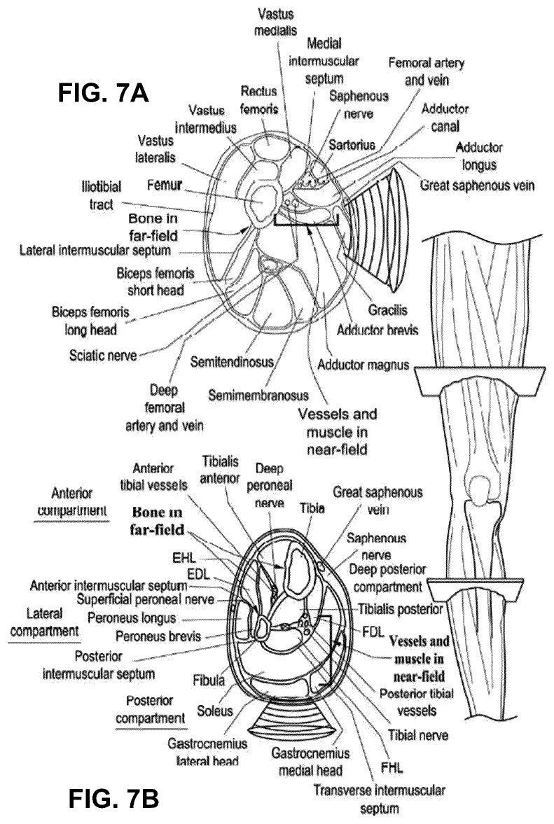

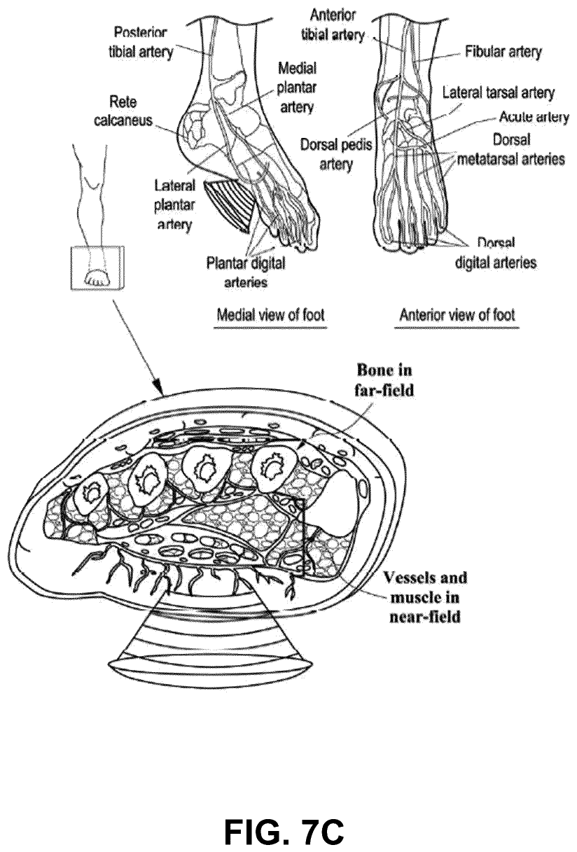

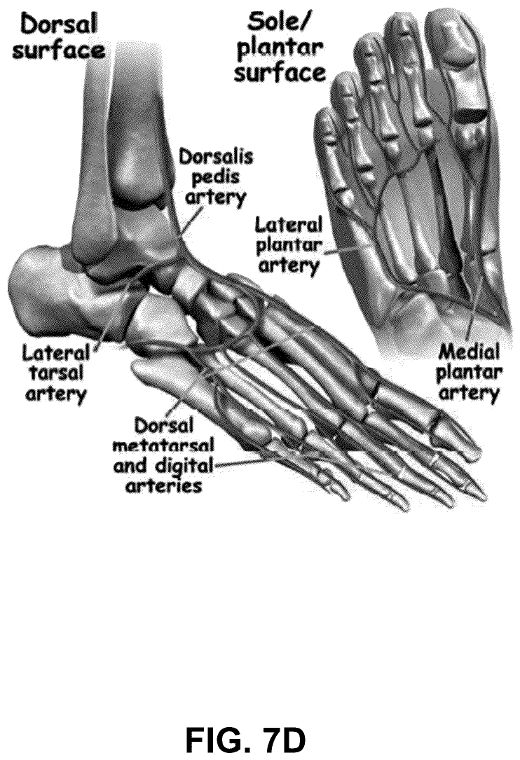

FIGS. 7A-7C illustrate non-limiting positions of an ultrasound transducer (e.g., transducer 106, 900, 901) or ultrasound transducer array (e.g., array 1000) adjacent the skin surface of an anatomical target location. Positioning of the transducer/array may be varied to optimize the effects of revascularization by placing arteries in the acoustic near-field, and to minimize acoustic reflection/scattering by maintaining bone in the far-field. In FIG. 7A, the transducer (or array) is illustrated positioned along the medial aspect of the thigh, maintaining the femoral artery in the acoustic near-field and femur in the far-field so as to maximize vascular exposure and minimize bone exposure of acoustic tissue. In FIG. 7B, the transducer (or array) is positioned over the posterior aspect of the calf, maintaining the gastrocnemius, soleus, and posterior tibial artery in the near-field, and tibia and fibula in the far-field. In FIG. 7C, the transducer (or array) is positioned over the plantar mid-foot, maintaining the plantar arterial arch in the near-field and metatarsals in the far-field. FIG. 7D schematically illustrate additional vessels on the dorsal and plantar surfaces of the foot that can be treated using systems and methods as disclosed herein.

Positioning of the transducers/array can be determined (such as by a healthcare provider) depending on the desired clinical result. In the thigh, the superficial femoral artery (SFA) is positioned anterior at the level of its bifurcation off of the common femoral artery, moves to the medial part of the thigh as it descends caudally, and rotates posteriorly where it becomes the popliteal artery. Thus, in some embodiments, to advantageously promote revascularization and/or condition vascular calcium in the SFA, the transducer/array can be positioned along the medial aspect of the thigh. In some embodiments, the transducer/array can also be positioned along the anterior, posterior, or lateral aspects of the thigh.

In the calf, the popliteal artery divides to become the anterior and posterior tibial arteries. The tibia is prominently situated along the anterior portion of the intrapopliteal lower extremity, and bone reflects and distorts acoustic energy. While the transducer/array can be positioned anteriorly, medially, or laterally, positioning the transducer/array along the posterior aspect of the calf can advantageously allow for increased revascularization and/or conditioning of vascular calcium in the posterior tibial artery with minimal acoustic distortion by the tibia and fibula.

In the ankle, the dorsalis pedis artery and the pedal arch run on the anterior surface of the ankle. Positioning of the transducer/array over the anterior surface can advantageously promote revascularization and/or conditioning of vascular calcium in these small vessels. Furthermore, the anterior surface of the ankle is relatively flat compared to the posterior surface, allowing for more secure positioning of the transducer/array. However, in some embodiments, the transducer/array can be positioned along the medial, lateral, or posterior surface of the ankle. The transducer/array can also be positioned underneath the foot, such as in the sole of a shoe or shoe insert for example.

In the foot, the plantar arch continues from the posterior tibial artery and bifurcates into the lateral and medial plantar arteries. The plantar foot also includes several layers of muscles and is relatively arched in a concave shape. Positioning of the transducer/array on or proximate the plantar surface of the foot can simultaneously promote revascularization and/or conditioning of vascular calcium in the plantar arch arteries.

Carotid, renal, and other arteries may be stenosed/obstructed in patients with or without PAD. In addition to the lower extremities, PAD patients often have atherosclerosis in the carotid arteries, placing them at risk for stroke and hypertension/renal injury, respectively. As with lower extremity PAD, treatment of two disease processes is typically also limited to medical therapy, and catheter-based or surgical revascularization, all of which have their limitations. AV fistulas for vascular access, for example, in patients undergoing hemodialysis, may also be treated with ultrasound to increase blood flow and preserve the AV fistula in some cases.

In some embodiments, an ultrasound-based device (e.g., wearable device 500) may be sized and configured to fit around the neck of a patient with a transducer on a desired location, e.g., unilateral or bilateral carotid arteries. The device can take the form of a collar, for example. The device can include a circular transducer (e.g., transducer 106, 900, 901), with a spherical curvature. The diameter of the transducer can be, for example, between about 1 cm and about 5 cm, such as between about 1 cm and about 2 cm, between about 2 cm and about 4 cm, and or between about 3 cm and about 5 cm. The transducer could have a radius of curvature, for example, of between about 0 cm and about 50 cm. In some embodiments, the transducer may be rectangular with a cylindrical curvature. The height of the transducer can be, for example, between about 1 cm and about 5 cm, such as between about 1 cm and about 2 cm, between about 2 cm and about 4 cm, or between about 3 cm and about 5 cm. The radius of curvature of the transducer can be, for example, between about 0 cm and about 100 cm. The width of the curvature can be, for example, between about 1 cm and about 5 cm, such as between about 1 cm and about 2 cm, between about 2 cm and about 4 cm, or between about 3 cm and about 5 cm.

In some embodiments, an ultrasound-based device (e.g., wearable device 500) may be sized and configured to fit around the torso, back, or abdomen with the transducer (e.g., transducer 106, 900, 901) positioned over one or both renal arteries. The device can include a circular transducer, with a spherical curvature. The diameter of the transducer can be, for example, between about 10 cm and about 30 cm, such as between about 10 cm and about 20 cm, between about 15 cm and about 25 cm, and or between about 20 cm and about 30 cm. The transducer can have a radius of curvature, for example, of between about 0 cm and about 300 cm. In some embodiments, the transducer can be rectangular with a cylindrical curvature. The height of the transducer can be, for example, between about 10 cm and about 30 cm, such as between about 10 cm and about 20 cm, between about 15 cm and about 25 cm, or between about 20 cm and about 30 cm. The radius of curvature of the transducer can be, for example, between about 0 cm and about 300 cm. The width of the curvature can be, for example, between about 10 cm and about 20 cm, such as between about 10 cm and about 15 cm, between about 15 cm and about 20 cm, between about 12 cm and about 15 cm, or between about 15 cm and about 18 cm.

In some embodiments, the wearable device could take the form of, include, or only include, for example, a vest (for the chest), armband (for the upper extremities), glove (for the hands), boot (for the ankle), sock (for the feet), a cup or undergarment (for vascular erectile dysfunction), decal/sticker (e.g., self-adhesive), abdominal or back brace/binder (e.g., for renal or abdominal indications) or other form factor depending on the desired clinical result.

### Example TUS System

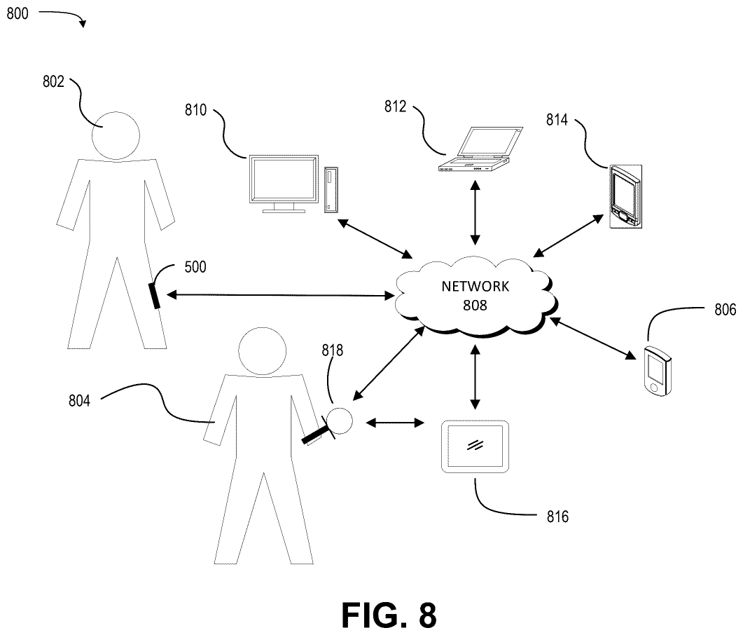

The TUS wearable device 500 can be used in a standalone manner and/or in combination with other devices and/or sensors. As shown in FIG. 8, the device 500 can be secured to the body of a subject (e.g., a patient) 802 and connect (for example, wirelessly) with a plurality of devices, including but not limited to a patient monitor 816 (for example, a bedside monitor, a patient monitoring and connectivity hub, any handheld patient monitoring devices, and any other wearable patient monitoring devices), a mobile communication device 806 (for example, a smartphone), a computer 810, 812 (which can be a laptop or a desktop), a tablet 814, glasses such as smart glasses configured to display images on a surface of the glasses, an imaging device 818 (for example, an ultrasound imaging probe), and/or the like. The wireless connection can be based on BLUETOOTH® technology, near-field communication (NFC) technology, and/or the like. Additionally, the wearable device 500 can connect to a computing network 808 (for example, via any of the connected devices disclosed herein, or directly). The network 808 may comprise a local area network (LAN), a personal area network (PAN), a metropolitan area network (MAN), a wide area network (WAN), or the like, and may allow geographically dispersed devices, systems, databases, servers (e.g., cloud-based), and the like to connect (e.g., wirelessly) and to communicate (e.g., transfer data) with each other.

Optionally, the device 500 can be integrated with one or more sensors and/or configured to connect to a plurality of external sensors, wirelessly or with a connecting cable. The connecting cable can be a universal connector configured to connect to any of the medical devices and/or sensors disclosed herein to provide communication between the wearable device 500 and the connected medical devices and/or sensors. The cable can optionally include a board-in-cable device that includes its own processor, but may not include its own display.

The wearable device 500 can include open architecture to allow connection of third-party wireless sensors and/or transducers, and/or allow third party access to a plurality of sensors and/or transducers on the wearable device 500 or connected to the wearable device 500. The plurality of sensors can include, for example, a temperature sensor, an altimeter, a gyroscope, an accelerometer, emitters, LEDs, etc. Third party applications can be installed on one or more devices shown in FIG. 8, such as the mobile communication device 806, and can use data from one or more of the sensors on the wearable device 500 and/or in electrical communication with the wearable device.

Optionally, the wearable device 500 can communicate with any other suitable noninvasive sensor, such as an acoustic sensor, a PPG sensor, a physiological sensor, a blood pressure sensor, temperature sensor, movement sensor, ECG sensor, etc. The wearable device 500 can optionally communicate with chemical sensors, which can detect, for example, chemicals on the user's skin, and/or sweat, and/or the odor of certain chemicals in the air. The chemical sensors can include electrochemical sensors or any other suitable types of chemical sensors.

In some embodiments, the wearable device 500 and imaging device 818 may be configured for image-guided TUS. In some aspects, the wearable device 500 and imaging device may be integrated as a single device configured to provide both ultrasound imaging and ultrasound therapy. For example, as further described herein, the imaging device may include one or more transducers configured for ultrasound imaging and one or more transducers (e.g., transducers 106, 900, 901) configured for delivering acoustic energy for TUS. The imaging device 818 may be implemented as an open-loop imaging device operating according to one or more preset parameters, or as a closed-loop imaging device for continuous monitoring of TUS and adjustment of imaging parameters based at least in part on received acoustic data. As further described herein, in some embodiments, the imagining device may be configured to transmit ultrasound image data for display in one or more devices shown in FIG. 8 for analysis and/or monitoring of TUS by a user 804 such as a healthcare provider. In some embodiments, the imaging device may be configured to (or be in communication with a device configured to) perform image analysis based at least in part on detected acoustic signatures and/or received acoustic data for continuous analysis and monitoring of TUS. Ultrasound/acoustic data can include data obtained after and/or based at least in part on reflection of generated acoustic waves by tissue and/or bone of an anatomical extremity.

The imaging device 818 may be a hand-held device, such as a hand-held ultrasound imaging probe, and/or may be secured to the subject 802. The subject 802 and/or the user (e.g., a healthcare practitioner) 804 may use the imaging device 818 to position a transducer (e.g., transducer 106, 900, 901) or transducer array (e.g., array 1000) and direct an acoustic field in a particular direction (e.g., beam steering), and set the focal depth of the therapeutic acoustic field. In some embodiments, the system/device can be configured to position the transducer/array and/or determine/select the direction of propagation of the acoustic field and/or set the focal depth via, for example, an algorithm based at least in part on image analysis. For example, as an arterial vessel is treated with TUS, various different portions of the vessel may have different locations and/or depths relative to the position of the transducer/array on the skin surface. Thus, various TUS parameters as described herein can be adjusted to ensure effective treatment of various different regions of vascular calcium as, for example, the device 500 targets different sections of the afflicted vessel using the transducer/array.

Advantageously, image guidance can help a user and/or system/device to identify and direct therapeutic acoustic energy to the point of treatment. Image-guided TUS can help ensure that transducers (e.g., transducer 106, 900, 901) are, for example, directing therapeutic acoustic energy to an intended treatment location by focusing an acoustic field and/or generating cavitation microbubbles at a target region of vascular calcium. For example, image guidance may help ensure that the acoustic field is focused, and/or cavitation microbubbles are generated, at the regions of the blood vessel that have hardening. Cavitation microbubbles can have high acoustic reflectivity. For example, cavitation clouds (e.g., a high density of cavitation microbubbles) can scatter incoming acoustic waves such as ultrasound waves for imaging. This can reflect acoustic energy to acoustic sensors, such as sensors 1212 described with reference to FIG. 9. Cavitation microbubbles can persist for a certain period of time after formation. In some embodiments, such microbubbles may persist for long enough duration after formation that they may be subsequently imaged by the system, such as by scattering/reflecting the ultrasound waves emitted by imaging transducers. Consequently, the microbubbles can appear in diagnostic ultrasound imaging. This may permit the system and/or a user to determine whether cavitation is being induced in the first place and/or whether cavitation is being induced at the correct/intended treatment location.

Advantageously, the system and/or a user may determine when next to cause one or more TUS transducers to emit acoustic waves for delivery of therapeutic energy to the target region based on the presence of cavitation microbubbles in the diagnostic images. If therapeutic energy is directed to the target region while microbubbles are present near or at the target region, the microbubbles may scatter and reflect the therapeutic energy, which can reduce (or minimize) therapeutic effects on the target region of vascular calcium. The system and/or user may cause the TUS transducers to emit acoustic waves for delivery of therapeutic energy to the target region based on a determination that the cavitation cloud has dissipated (e.g., a plurality of, a majority of, or all microbubbles have burst and no longer appear in diagnostic images). For example, after the cavitation cloud has dissipated, the system and/or user may cause the TUS transducers to emit acoustic waves for delivery of therapeutic energy to the target region. This can reduce (or minimize or eliminate) said scattering effects and help ensure effective delivery of therapeutic energy to the target region of vascular calcium.

Examples of imaging techniques that may be implemented via the system 800 include acoustic radiation force imaging (ARFI), tissue elastography, and the like. In some embodiments, TUS can be monitored by analyzing blood flow in targeted arterial vessels. For example, after conditioning vascular calcium, blood flow through the vessel may increase, and the increase in blood flow can be imaged such as by imaging device 818. In some examples, various imaging techniques, including but not limited to Doppler ultrasound, may be implemented by the imaging device 818 to determine blood flow through the targeted vessel. In some examples, a separate Doppler ultrasound device may be used.

Moreover, image guidance can advantageously help ensure that the level of TUS is safe by, for example, determining whether the TUS device is softening vascular calcium sufficient for revascularization or subsequent PAD interventions, but not imparting so much acoustic energy that vascular calcium is dislodging from vessel walls/surfaces and flowing downstream to occlude the vessel (e.g., becoming thrombotic). In some embodiments, the imaging device 818 may monitor for vascular injury.

Continuous analysis and/or monitoring of TUS treatment can include determining TUS parameters while continuously measuring acoustic data, and/or monitoring such data in real-time. Continuous monitoring can include obtaining acoustic data (e.g., one or more signals indicative of a TUS parameter) from one or more sensors (such as any of the sensors disclosed herein, e.g., acoustic sensor) at a sampling rate sufficient to consistently capture irregular physiological events while the patient/subject is in contact with the wearable device. Continuous monitoring may include sampling acoustic data at a sampling rate until the wearable device identifies a likeliness of a physiological event and then may adjust the sampling rate (e.g., increase or decrease the sampling rate) to better capture acoustic data regarding the event.

Such continuous measurement/monitoring may allow for monitoring physiological trends and/or for generating a smoother waveform based on the acoustic data, which may provide additional information in regard to corresponding TUS parameters. A sampling rate used for continuous measurement may be determined, in some examples, based on a rate at which meaningful changes in acoustic data are reasonably anticipated. In some examples, a sampling rate can be defined by relatively short time periods such that even minor changes in the acoustic data over time are captured. For example, continuous measurement may include obtaining acoustic data at a sampling rate of 1 minute, 30 seconds, 24 seconds, 12 seconds, 11 seconds, 10 seconds, 9 seconds, 8 seconds, 7 seconds, 6 seconds, 5 seconds, 4 seconds, 3 seconds, 2 seconds, 1 second, 0.5 seconds, or 0.1 seconds. A continuous sampling rate may, in some examples, be selected or updated based on anticipated changes in monitored acoustic data, sensor type, TUS parameter being monitored or tracked, hardware considerations (e.g., battery life), or physiological or hardware considerations.

Monitoring acoustic data in real time may include processing, transmitting, and/or displaying such data within a short time period after such data is obtained, for example, within 10 seconds, within 5 seconds, within 4 seconds, within 3 seconds, within 2 seconds, within 1 second, within 0.9 seconds, within 0.7 seconds, within 0.5 seconds, within 0.3 seconds, or within 0.1 seconds from when such data is obtained. Real time monitoring, especially when paired with continuous measurement, can facilitate improved monitoring of physiological conditions of the user that may change in relatively short time periods. This can in turn facilitate accurate detection of changes in physiological condition and rapid response to such changes (if necessary).

Continuously monitored acoustic data and TUS parameters by the device (e.g., device 818, 500) can be beneficial to the patient/subject. Continuous monitoring can advantageously improve detection of critical events such as thrombosis, indications of damage to blood vessels, and/or the like. Continuous monitoring can advantageously allow for early detection of vascular abnormalities such as vascular malformations and/or or the like. Continuous monitoring can enable timely adjustment of TUS treatment and/or medical intervention, which can be crucial in preventing complications or worsening of a physiological condition. Continuous monitoring can lead to improved management of TUS treatment by, for example, keeping track of changes in acoustic data so that users of the wearable device can be effective and safe in delivering TUS therapy. Advantageously, continuous monitoring can help to tailor TUS solutions to individual needs based on, for example, personal acoustic data trends.

FIG. 9 depicts a block diagram illustrating an example implementation of a device 1200 of TUS system 800. The computing device 1200 can be implemented as, integrated into, and/or include any of the features and/or functions of any of the devices described herein (e.g., wearable device 500, imaging device 818, etc.), such as in FIG. 8. Computing device 1200 may be a separate device operably connected to and in communication (e.g., transfer data) with any of the devices described herein. The computing device 1200 can include one or more hardware processors 1202, a memory 1204, a communication interface 1206, a power source 1208, one or more transducers 1210, one or more sensors 1212, and a display 1214.

The hardware processor 1202 can be configured to execute program instructions to cause the computing device 1200 to perform one or more operations. The hardware processor 1202 can be configured, among other things, to process data (e.g., analyze, cleanse, edit, reduce, wrangle, or otherwise process data), execute instructions to perform one or more functions, and/or control the operation of the computing device 1200 or components thereof. For example, the hardware processor 1202 can process sensor data obtained from sensor(s) such as acoustic sensors (e.g., sensor(s) 1212) and can execute instructions to perform functions related to storing and/or transmitting such sensor data. In some examples, the hardware processor 1202 can process data received from one or more devices described herein, such as shown and/or described in FIG. 8. In some embodiments, the hardware processor 1202 can be configured to perform one or more operations based on user input received via, for example, a user interface or other device (e.g., a device in communication with computing device 1200). For example, the hardware processor may cause one or more transducers (e.g., transducers 106, 900, 901) to emit acoustic waves at a certain frequency, for a certain period of time, and/or of a certain acoustic intensity based on received user input (such as any frequency, treatment time, and/or acoustic intensity described herein). In some examples, the hardware processor 1202 can cause one or more transducers (e.g., transducers 106, 900, 901) to operate according to a certain duty cycle, such as any of the duty cycles described herein, based on received user input. In some aspects, the hardware processor 1202 may be remote to the computing device 1200.

The memory 1204 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. Such stored data can be processed and/or unprocessed data obtained from sensor(s) such as acoustic sensors (e.g., sensor(s) 1212). Stored data can be processed and/or unprocessed data obtained from one or more devices described herein, such as shown and/or described in FIG. 8. Stored data may include time-stamped data, including but not limited to time instants and time durations of TUS treatments to permit a healthcare provider to monitor compliance. This may be done through connection with a TUS device for download of usage data, wireless synchronization to handheld devices or clinical remote monitoring devices, and/or the like, via the communication interface 1206, for example. Stored data can include user input such as acoustic/TUS parameters selected by a user via, for example, a user interface. Synchronization with a device, such as a handheld device or other computing device, may allow a user to enter acoustic/TUS parameters to be transmitted to the computing device 1200. The memory 1204 can store program instructions that when executed by the hardware processor 1202 cause the computing device 1200 to perform one or more operations.

Memory 1204 can store persistent data and/or non-persistent data. Persistent data may be data that is preserved (e.g., not deleted from storage) when the computing system is powered down and/or when an application is terminated. Persistent data may be stored for longer than 30 seconds, longer than 60 seconds, longer than 5 minutes, longer than 10 minutes, longer than 30 minutes, longer than 1 hour, longer than 6 hours, longer than 12 hours, longer than 24 hours, or the like. Non-persistent data may be data that is deleted or otherwise lost when the computing system is powered down and/or when an application is terminated. Non-persistent data may be stored for less than 24 hours, less than 12 hours, less than 6 hours, less than 1 hour, less than 30 minutes, less than 10 minutes, less than 5 minutes, less than 60 seconds, less than 30 seconds, less than 20 seconds, less than 10 seconds, less than 5 seconds, less than 1 seconds, or the like. Non-persistent data may be stored for a shorter period of time than persistent data. In some implementations, persistent data may be stored in non-volatile memory. In some implementations, non-persistent data may be stored in volatile memory such as RAM. Memory 1204 can store data in a buffer. A buffer may store data for a period of time before deleting the data. The period of time can be fixed. The buffer may automatically delete data stored therein upon expiration of a period of time. The period of time may be between about 0.01 seconds and 0.15 seconds, between 0.1 seconds and 1.5 seconds, between 1 second and 5 seconds, between 1 second and 10 seconds, between 10 seconds and 60 seconds, between 30 seconds and 60 seconds, between 1 minute and 3 minutes, between 1 minute and 5 minutes, between 1 minute and 10 minutes, between 5 minutes and 30 minutes, between 20 minutes and 60 minutes, or greater than 60 minutes.

The communication interface 1206, which may also be referred to as a communication system, can facilitate communication (via wireless, wired, and/or wire-like connection) between the computing device 1200 (and/or components thereof) and separate devices, such as separate monitoring hubs, monitoring/wearable TUS devices, sensors, imaging devices, user devices, systems, servers, and/or the like. For example, the communication interface 1206 can be configured to allow the computing device 1200 to wirelessly communicate with other devices, systems, and/or networks over any of a variety of communication protocols such as described herein, including near-field communication protocols and far-field communication protocols. Near-field communication protocols, which may also be referred to as non-radiative communication, can implement inductive coupling between coils of wire to transfer energy via magnetic fields (e.g., NFMI). Near-field communication protocols can implement capacitive coupling between conductive electrodes to transfer energy via electric fields. Far-field communication protocols, which may also be referred to as radiative communication, can transfer energy via electromagnetic radiation (e.g., radio waves). The communication interface 1206 can communicate via any variety of communication protocols such as WI-FI®, BLUETOOTH®, ZIGBEE®, Z-WAVE®, cellular telephony, 1G, 2G, 3G, 4G, 5G, infrared, radio frequency identification (RFID), satellite transmission, inductive coupling, capacitive coupling, proprietary protocols, combinations of the same, and the like. The communication interface 1206 can allow data and/or instructions to be transmitted and/or received to and/or from the computing device 1200 and separate computing devices. The communication interface 1206 can be configured to transmit and/or receive (for example, wirelessly) processed and/or unprocessed data with separate computing devices including monitoring hubs, TUS wearable devices, sensors, systems, imaging devices, user devices, remote servers, and/or the like. In some embodiments, communication interface 1206 can transfer power required for operation of a computing device.

In some embodiments, the communication interface 1206 can be embodied in one or more components that are in communication with each other. The communication interface 1206 can include one or more of: transceivers, antennas, transponders, radios, emitters, detectors, coils of wire (e.g., for inductive coupling), and/or electrodes (e.g., for capacitive coupling). The communication interface 1206 can wirelessly communicate or connect to one or more remote computing devices over a network such as by implementing one or more wireless communication protocols.

The power source 1208 can provide power for the computing device 1200 or components thereof. Power source 1208 can include a battery and can be rechargeable. In some embodiments, the power source 1208 may be external to the computing device 1200. For example, the computing device 1200 can include or can be configured to connect to a cable which can itself connect to an external power source to provide power to the computing device 1200. The power source 1208 may be a portable battery. The power source 1208 can include a plurality of batteries. The power source 1208 can additionally or alternatively be configured to be solar powered and charged, for example, by including a solar panel assembly on the computing device 1200 and/or power source 1208.

In some embodiments, power source 1208 can be embodied in one or more components that are in communication with each other. For example, power source 1208 may be operably connected to, or integrated into, transducers/transducer arrays to allow programming for pulsed or continuous wave TUS. For example, the power source 1208 may be operably connected to, or integrated into, a generator/interface console that includes one or more features and/or functions of computing device 1200.

Connection to an external power source can be high risk in some cases and uncomfortable for nighttime use. A portable, detachable, and rechargeable battery (with incorporated generator) can be connected to one or more transducers, and also fixed into position within a TUS wearable device (device 500, 818, 1200). During times of non-use, the battery can be disconnected from transducers, removed from device, and connected to a charging station. Battery capacitance/voltage design can be sufficient in some cases to power a device for 8 or more hours of use or another appropriate time period at aforementioned TUS parameters. Some embodiments can include wired AC power connections.

Transducer(s) 1210 can emit acoustic waves that propagate toward a region of vascular calcium. Transducer(s) 1210 may be configured to emit acoustic waves according to any of the TUS parameters described herein, including but not limited to acoustic waves having a frequency from about 400 kHz to about 5 MHz, a pulse duration from about 5 μs to about 100 μs, pulse repetition frequency from about 0.5 Hz to about 2000 Hz, peak negative pressure from about 4 MPa to about 24 MPa. The computing device 1200 may cause one or more transducers 1210 to pulsatile or continuous acoustic waves for TUS treatment durations from about 1 minute to 120 minutes. Such TUS treatment may be repeated at multiple intervals.

Transducer(s) 1210 may be implemented as and/or include one or more functions and/or features of transducers 106, 900, 901. For example, transducer(s) 1210 may be configured to emit acoustic waves at frequencies effective for TUS treatment, such as at frequencies from about 400 kHz to about 2 MHz. As described herein, acoustic frequencies within the aforementioned range can be conducive to generating a high density of cavitation microbubbles (e.g., a cavitation cloud) at a region of vascular calcium to induce localized shockwaves at the level of the calcium when the microbubbles collapse.

In some examples, transducer(s) 1210 may emit acoustic waves in a frequency range effective for ultrasound imaging, such as from about 3 MHz to about 12 MHz.

In some embodiments, the computing device 1200 (e.g., TUS wearable device 500, imaging device 818) can include transducers configured for TUS treatment and/or imaging. In some embodiments, transducer(s) 1210 may be external of the computing device 1200, and, for example, may be integrated into, housed by, or connected to a separate wearable device 500 and/or imaging device 818. For example, a wearable or image device such as described herein may include an array of transducers configured for TUS and an array of transducers configured for imaging. In some examples, an array of therapeutic transducers may be coaxial or collinear with an array of imaging transducers.

In some aspects, for delivery of therapeutic energy during treatment, hardware processor 1202 may send one or more control signals to one or more transducers causing one or more transducers, such as a first subset of transducers, to emit acoustic waves in a frequency range effective for TUS treatment. The hardware processor 1202 may send one or more control signals to one or more transducers turning off or prohibiting one or more transducers, such as a second subset of transducers, from emitting acoustic waves in a frequency range effective for imaging. In some aspects, for imaging of an anatomic region (e.g., a targeted region of vascular calcium), hardware processor 1202 may send one or more control signals to one or more transducers causing one or more transducers, such as the second subset of transducers, to emit acoustic waves in a frequency range effective for imaging. The hardware processor 1202 may send one or more control signals to one or more transducers turning off or prohibiting one or more transducers, such as the first subset of transducers, from emitting acoustic waves in a frequency range effective for TUS treatment. In some embodiments, the first subset of transducers may be different than the second subset of transducers. In some embodiments, one or more transducers may be configured to emit acoustic waves for TUS at a first time period and emit acoustic waves for imaging at a second time period.

In some embodiments, as further describe herein, the computing device 1200 (or any other device described herein) may determine to switch between causing one or more transducers to emit acoustic waves for delivery of therapeutic energy and imaging based on, for example, diagnostic ultrasound-based signatures that may trigger delivery of TUS. In some embodiments, a user (e.g., a healthcare provider) may manually switch between causing one or more transducers to emit acoustic waves for delivery of therapeutic energy and acoustic waves for imaging (e.g., via pressing a button of the device).

Sensor(s) 1212 can provide sensor data to the computing device 1200 or components thereof. Sensor(s) 1212 can include any of the sensors described herein, including but not limited to acoustic sensors, temperature sensors, altimeters, gyroscopes, accelerometers, emitters, LEDs, physiological sensors, chemical sensors, etc. In some embodiments, sensor(s) 1212 can be noninvasive sensors. In some embodiments, sensor(s) 1212 can be external of the computing device 1200. For example, sensor(s) 1212 may be integrated into, housed by, or coupled to another device, such as any device described herein. In some embodiments, sensor(s) 1212 such as acoustic sensors may be integrated into, housed by, or connected to transducer(s) 1210.

Sensor(s) 1212 may be configured to measure an acoustic parameter in real time as transducers emit acoustic waves for delivery of therapeutic energy or imaging. Acoustic parameters can include any of the parameters described herein. In some aspects, sensor(s) 1212 may be in communication with one or more hardware processors 1202. The hardware processor 1202 can be configured to process one or more signals (e.g., acoustic signals) originating from the one or more sensors 1212 to determine a measured parameter (e.g., acoustic parameter). In some aspects, the hardware processor 1202 can be configured to modify TUS treatment (e.g., modify TUS parameters) in real-time. The hardware processor 1202 can be configured to modify TUS treatment based at least in part on signals such as acoustic signals received from sensor(s) 1212 as described herein. The hardware processor 1202 can be configured to modify TUS treatment based at least in part on data obtained from other devices as described herein.

Modifying TUS treatment can include modifying TUS parameters, such as adjusting (increasing or decreasing) or keeping same acoustic frequency, treatment time, duty cycle, acoustic intensity, and other parameters described herein. The computing device 1200 may be configured to implement a closed-loop operation such that TUS parameters are continuously monitored and/or updated based on detected signals obtained from sensor(s) 1212 via, for example, an algorithm. In some aspects, hardware processor 1202 may modify one or more TUS parameters according to various different focal depths corresponding to various different sections of a target vessel, which may be disposed at different depths with respect to the transducer(s) 1210. For example, sensor(s) 1212 can include a time-of-flight sensor that determines time duration for a reflected acoustic signal to impinge upon the sensor, after which the hardware processor 1202 can determine the depth of the portion of vessel that reflected the emitted acoustic wave.

Particularly with prolonged nighttime use (e.g., up to 12 hours), at higher acoustic intensities (p-, Ispta), and longer duty cycles (e.g., greater than 10%), heating of the transducer and skin may occur. Typically, FDA requirements require a thermal index of less than 6.0, and a probe surface not to exceed 43° C. in contact with skin, and 50° C. in air. Incomplete seal with ultrasound gel or other adhesives resulting in significant air bubbles in the acoustic field may increase heating. Thus, safety mechanisms can be beneficial to prevent thermal skin damage. In some embodiments, a thermocouple may be integrated onto the transducer surface with a feedback loop to the battery/generator to turn off the device upon sensing a temperature that satisfies (e.g., is greater than) a predetermined threshold (e.g., greater than about 40° C., 41° C., 42° C., 43° C., or more or less in some embodiments) via one or sensors 1212 (e.g., temperature sensors). In some embodiments, the device including the transducer (or transducer array) can include a cooling system to prevent overheating and temperature control and may be cooled via, for example, a fluid, such as in a closed fluid loop that circulates around the transducer to remove heat. In some embodiments, the transducer may be air-cooled via one or more fans. In some embodiments, ultrasound gels with large heat capacitance can be utilized. The gels may include, for example, a conformable, high heat-capacity matrix with embedded thermal capacitors comprising phase change materials (PCMs) or other endothermic materials. In some embodiments, the thermal conductivity of the PCMs or the gel itself may be enhanced through the addition of high thermal conductivity particles. These particles can include materials such as, for example, thermally conducting polymers, metallic nano or micro particles, carbon-based materials, or other high thermal conductivity materials. In some embodiments, a sleeve, band, vest, socks (or other wearable transducer/array holder) can include cut-out windows, or be made of a breathable material for air cooling of the transducer. An adhesive coupling gel pack between the transducer and skin can also help to dissipate transducer heat by conduction, and limit heating of skin. In some embodiments, systems and methods can involve sensing the impedance at the skin surface and decreasing or terminating the ultrasonic energy delivery if the impedance satisfies (e.g., is greater than) a pre-determined threshold.

In some embodiments, adequate and gasless coupling of the transducer/array to skin can be monitored by real-time, in-treatment assessment of reflected acoustic power back at the transducer via one or more sensors 1212 (e.g., acoustic sensors). High reflected power (e.g., about or at least about 5%, 6%, 7%, 8%, 9%, 10%, 11%, 12%, 13%, 14%, 15%, or more of the forward power delivered by the generator) can be indicative of air in the transducer-skin interface, and the computing device 1200 can be programmed to automatically stop energy delivery in such cases.

In some embodiments, each transducer 1210 can be driven by a short pulse length with a long duty cycle as the control activation cycles through each of the transducers, only one at a time in some cases. In some embodiments, to confirm proper coupling of the transducer to the skin, a measurement of power delivered on each transmit pulse can be used. Confirmation of proper coupling may be implemented via digital signal processing. For example, the drive voltage can be measured on both sides of a reference source resistor, such as via sensors. Both signals can be simultaneously sampled to acquire a large amount of data (for example, 2048 samples at 20 MHz for a 100 μs sample in the middle or in the latter half, such as third or fourth quarter for example of the 1000 μs nominal pulse length). Each signal can be quadrature demodulated to baseband and summed (e.g., a single frequency DFT). Amplitude and phase can then be calculated for each signal. With the source resistance and the amplitude and phase from each signal, the drive voltage can be calculated, along with the drive current and relative phase between them. From this, the electrical power delivered can be calculated. With reference power measurements into water (perfect coupling) and air (no coupling), the system can determine if each transducer is coupled to the patient and operating properly. If less power, such as below a predetermined threshold (perhaps due to poor coupling) is detected by the electrical power measurement, the system can turn off and alert the user via a visual, auditory, tactile/haptic, or other indication/alarm.

Bone, implants (e.g., titanium implants), external braces or solid matter or other hardware, or other highly echogenic material in the near-field of the transducer can be undesirable, and may lead to acoustic reflection, scattering, and may increase risk of transducer heating or other adverse events. Thus, in some embodiments, the device may include one or more ultrasound or non-ultrasound (e.g., photo or video based, such as a CCD, CMOS, or other camera configured to identify the target anatomy, X-ray, CT, or MR based) imaging components (e.g., ultrasonic/acoustic sensors or software/image processing quantification of echogenicity) to assess for echogenic bone or air in the near-field. This may be incorporated into the therapeutic transducers themselves (A-mode imaging), or with a separate imaging transducer (M-mode or B-mode imaging). When not applying therapeutic ultrasound, for example, therapeutic transducers in diagnostic mode or separate diagnostic imaging transducers can send diagnostic ultrasonic energy (e.g., in pulses) to measure reflected acoustic power/energy (or other acoustic features, characteristics, and the like). As described hereinabove, the device can include dual diagnostic imaging and therapeutic functions, and alternate diagnostic ultrasound with therapeutic ultrasound.

In some embodiments, the diagnostic imaging modality can be utilized to locate and identify the particular anatomy to be treated (e.g., with anatomical landmarks identifiable by the device). As such, in some embodiments, systems and methods can advantageously direct therapeutic energy to target tissue (e.g., regions of vascular calcium), and reduce (or minimize) energy delivery to bone or other solid/relatively more echogenic tissues. As further described herein, the system may be configured to obtain acoustic and/or image data associated with a targeted anatomic region (such as for TUS treatment and/or imaging) to ascertain whether the targeted anatomic region is in fact vascular calcium. For example, the system may determine various different acoustic features, structural characteristics, and the like associated with the targeted anatomic region. Additionally, or alternatively, the system may generate and output a visualization of the targeted anatomic region for viewing by the user.

In some embodiments, imaging may include intravenous or intraarterial injection of ultrasound image contrast agents, such as microbubble contrast agents, to enhance resolution. Advantageously, such microbubble contrast agents may reduce the threshold peak negative pressure required to generate sufficient cavitation at a region of vascular calcium. For example, therapeutic energy emitted by transducers may cause the contrast agents to burst and impart localized shockwaves at the level of vascular calcium, which can reduce the number of additional cavitation microbubbles needed to be generated by the acoustic waves themselves.

Detection of blood vessels and/or regions of vascular calcium may include determining a level of reflected acoustic energy and comparing the reflected acoustic energy to an acoustic energy threshold, such as a predetermined acoustic energy threshold. The threshold can include limits such as lower and upper limits. The hardware processor 1202 may be configured to determine a change in the reflected acoustic energy between a first time and a second time. The hardware processor 1202 may be configured to determine whether a targeted anatomical region (e.g., a region at which emitted acoustic waves are directed) is an arterial vessel and/or region of vascular calcium based on whether a value, a change, a rate of change, or a change in the rate of change of the reflected acoustic energy satisfies a threshold and/or satisfies a threshold a certain number of times.

For example, hardware processor 1202 may be configured to determine a level of reflected acoustic energy, such as a reflected acoustic energy level value, based on acoustic data received from one or more acoustic sensors. The hardware processor 1202 can be configured to determine whether a targeted anatomic region is an arterial vessel and/or region of vascular calcium based on a determination of whether the determined reflected acoustic energy level value satisfies the acoustic energy threshold. In some examples, the hardware processor 1202 may determine that a targeted anatomic region is an arterial vessel and/or region of vascular calcium based on a determination that the determined reflected acoustic energy level value is at or less than a predetermined reflected acoustic energy level value. In some examples, the hardware processor 1202 may determine that the targeted anatomic region is not an arterial vessel and/or region of vascular calcium (e.g., is echogenic bone or air) based on a determination that the determined reflected acoustic energy level value is greater than the predetermined reflected acoustic energy level value.

Acoustic energy thresholds may be fixed or programmable. In some aspects, acoustic energy thresholds can be set and/or modified by the wearer/user utilizing any device described herein. In some aspects, acoustic energy thresholds can be set and/or modified by the wearer/user utilizing the TUS wearable device (e.g., device 500) or a non-wearable device via a device application (e.g., a smart watch application, a smartphone application, etc.) or a web-based application. In some embodiments, acoustic energy thresholds may be set based on known normal parameters for correct device positioning.

In some embodiments, the hardware processor 1202 may be configured to determine a ratio of reflected energy, such as according to the following equation:

$$\begin{matrix}R = \\ \frac{{E}_{reflected}}{{E}_{transmitted}} ,\end{matrix}$$

wherein R is the ration of reflected energy, E_{reflected} is the level of acoustic energy of acoustic waves that return to the TUS device after reflection, and E_{transmitted} is the level of acoustic energy delivered by the TUS device. The hardware processor 1202 may be configured to compare the ratio R to a predetermined ratio R, such as a predetermined ratio threshold. The predetermined ratio threshold can include limits such as lower and upper limits. The hardware processor 1202 may be configured to determine a change in the ratio of reflected acoustic energy between a first time and a second time. The hardware processor 1202 may be configured to determine whether a targeted anatomical region (e.g., a region at which emitted acoustic waves are directed) is an arterial vessel and/or region of vascular calcium based on whether a value, a change, a rate of change, or a change in the rate of change of the reflected acoustic energy satisfies a threshold and/or satisfies a threshold a certain number of times.

For example, hardware processor 1202 may be configured to determine a ratio of reflected acoustic energy based on acoustic data received from one or more acoustic sensors. The hardware processor 1202 can be configured to determine whether a targeted anatomic region is an arterial vessel and/or region of vascular calcium based on a determination of whether the determined ratio of reflected acoustic energy satisfies the ratio threshold. In some examples, the hardware processor 1202 may determine that the targeted anatomic region is an arterial vessel and/or region of vascular calcium based on a determination that the determined ratio of reflected acoustic energy is at or less than a predetermined ratio of reflected acoustic energy. In some examples, the hardware processor 1202 may determine that the anatomical region being imaged is not an arterial vessel and/or region of vascular calcium (e.g., is echogenic bone or air) based on a determination that the determined ratio of reflected acoustic energy is greater than the predetermined ratio of reflected acoustic energy.

Ratios of acoustic energy may be fixed or programmable. In some aspects, ratios of acoustic energy can be set and/or modified by the wearer/user utilizing any device described herein. In some aspects, ratios of acoustic energy can be set and/or modified by the wearer/user utilizing the TUS wearable device (e.g., device 500) or a non-wearable device via a device application (e.g., a smart watch application, a smartphone application, etc.) or a web-based application. In some embodiments, ratios of acoustic energy may be set based on known normal parameters for correct device positioning.

In some embodiments, reflected acoustic energy may correspond to a voltage detected at the transducer. For example, the hardware processor 1202 may be configured to determine a voltage level (e.g., a voltage level value) indicative of the detected voltage and corresponding to the reflected acoustic energy, and compare the voltage level to a voltage threshold, such as a predetermined voltage threshold. The threshold can include limits such as lower and upper limits. The hardware processor 1202 may be configured to determine a change in the voltage level between a first time and a second time. The hardware processor 1202 may be configured to determine whether a targeted anatomical region (e.g., a region at which emitted acoustic waves are directed) is an arterial vessel and/or region of vascular calcium based on whether a value, a change, a rate of change, or a change in the rate of change of the voltage level satisfies a threshold and/or satisfies a threshold a certain number of times.

For example, the hardware processor 1202 can be configured to determine whether a targeted anatomical region is an arterial vessel and/or region of vascular calcium based on a determination of whether the determined voltage level value satisfies the voltage threshold. In some examples, the hardware processor 1202 may determine that the targeted anatomic region is an arterial vessel and/or region of vascular calcium based on a determination that the determined voltage level value is at or less than a predetermined voltage level value. In some examples, the hardware processor 1202 may determine that the targeted anatomic region is not an arterial vessel and/or region of vascular calcium (e.g., is echogenic bone or air) based on a determination that the determined voltage level value is greater than the predetermined voltage level value.

Voltage thresholds may be fixed or programmable. In some aspects, voltage thresholds can be set and/or modified by the wearer/user utilizing any device described herein. In some aspects, voltage thresholds can be set and/or modified by the wearer/user utilizing the TUS wearable device (e.g., device 500) or a non-wearable device via a device application (e.g., a smart watch application, a smartphone application, etc.) or a web-based application. In some embodiments, voltage thresholds may be set based on known normal parameters for correct device positioning.

In some embodiments, bone or other undesired material can be identified by the system (e.g., one or more devices, hardware processor 1202) based on differential acoustic reflectivity. For example, bone may have an acoustic reflectivity that is about 1.5x, 1.75x, 2x, 2.25x, 2.5x, 2.75x, 3x, or more greater than background or average reflectivity. The system may identify bone based on a predetermined differential acoustic reflectivity, and direct therapeutic energy to the near-field away from the bone or other undesirable material.

In some embodiments, detection of arterials vessels and/or vascular calcium may be based at least in part on detected blood flow, such as via Doppler imaging. As described hereinabove, doppler imaging may be implemented by the device, or a separate dedicated Doppler imaging device can be used in combination with the TUS device. Doppler imaging may be utilized to determine a presence/absence of blood flow in an imaged anatomic region and/or estimate a flow rate of blood in the imaged anatomic region. In some embodiments, detected blood flow may be indicative of a presence of arterial vessels, whereas detected absence of blood flow may be indicative of a presence of hard tissue, such as bone. For example, blood flows through arterial vessels, whereas blood does not flow through bone. In some embodiments, detected blood flow may be indicative of a presence of vascular calcium. For example, blood can flow (often at reduced flow rate compared to that of arterial vessels having no vascular calcium) through arterial vessels that are afflicted with calcium buildup.

In some embodiments, detection of vascular calcium may include determining a blood flow rate and comparing the determined flow rate to a flow rate threshold, such as a predetermined flow rate threshold. The threshold can include limits such as lower and upper limits. The hardware processor 1202 may be configured to determine a change in the flow rate between a first time and a second time. The hardware processor 1202 may be configured to determine whether a targeted anatomic region (e.g., a region at which emitted acoustic waves are directed) is an arterial vessel and/or region of vascular calcium based on whether a value, a change, a rate of change, or a change in the rate of change of the flow rate satisfies a threshold and/or satisfies a threshold a certain number of times.

For example, hardware processor 1202 may be configured to determine a blood flow rate, such as a blood flow rate value, based on acoustic data received from one or more acoustic sensors. The hardware processor 1202 can be configured to determine whether a targeted anatomic region is an arterial vessel and/or region of vascular calcium based on a determination of whether the determined flow rate value satisfies the flow rate threshold. In some examples, the hardware processor 1202 may determine that the targeted anatomic region is a region of vascular calcium based on a determination that the determined flow rate value is less than a predetermined flow rate value. In some examples, the hardware processor 1202 may determine that the targeted anatomic region is not a region of vascular calcium based on a determination that the determined flow rate value is at or greater than the predetermined flow rate value.

Flow rate thresholds may be fixed or programmable. In some aspects, flow rate thresholds can be set and/or modified by the wearer/user utilizing any device described herein. In some aspects, flow rate thresholds can be set and/or modified by the wearer/user utilizing the TUS wearable device (e.g., device 500) or a non-wearable device via a device application (e.g., a smart watch application, a smartphone application, etc.) or a web-based application. In some embodiments, flow rate thresholds may be set based on known normal parameters for arterial blood flow.

In some embodiments, the system can differentiate between regions of vascular calcium and other hard tissues (e.g., bone) based at least in part on determined characteristics of the anatomic structure of the targeted anatomic region. For example, the hardware processor 1202 may be configured to determine the size of the anatomic structure of the targeted anatomic region based on detected acoustic signatures and compare the structure size to a size threshold. The threshold can include limits such as lower and upper limits. In some embodiments, the threshold limits may correspond to the size of one or more bones (e.g., the smallest bone) in the anatomical extremity. In some embodiments, the hardware processor 1202 may determine that a region of hard tissue is vascular calcium and not bone based on a determination that the structural size of the imaged region satisfies the size threshold. For example, hardware processor 1202 may determine that a region of hard tissue is vascular calcium and not bone based on a determination that the determined structural size of the imaged region is less than a predetermined size threshold value. In some examples, the hardware processor 1202 may determine that a region of hard tissue is bone and not vascular calcium based on a determination that the determined structural size of the imaged region is at or greater than the predetermined size threshold value.

Size thresholds may be fixed or programmable. In some aspects, size thresholds can be set and/or modified by the wearer/user utilizing any device described herein. In some aspects, size thresholds can be set and/or modified by the wearer/user utilizing the TUS wearable device (e.g., device 500) or a non-wearable device via a device application (e.g., a smart watch application, a smartphone application, etc.) or a web-based application. In some embodiments, size thresholds may be set based on the extremity being targeted for TUS treatment and/or imaging and known bone sizes in the respective extremity. For example, the smallest bone in the lower extremities (the fibula) can be larger than any arterial vessel (and thus larger than any region of vascular calcium) of the lower extremities.

In some embodiments, detection of blood vessels and/or regions of vascular calcium can be based at least in part on anatomic locations (e.g., known anatomic locations) of arterial vessels and other hard tissue (e.g., bone) and surrounding structures in the anatomical region. Calcification of blood vessels may usually occur along known vascular pathways (e.g., the various peripheral arteries), such as along and/or within the lumen of arterial vessels. Arterial vessels are often located near soft tissue structures such as muscles, ligaments, and/or tendons. Hard tissue such as bones often appear in predictable places, such as near joints (e.g., the knee, the ankle, the elbow, the wrist, etc.), and along major skeletal landmarks (e.g., femur, spine, ribs, pelvis, etc.). The system may be configured to determine whether a target region is vascular calcium or bone based on surrounding anatomic regions, such as the target region being located proximate to a skeletal landmark or joint. In some embodiments, the system may provide a user with a visualization of the targeted anatomic region (e.g., via imaging transducers), and the user may provide input to the system indicating that the particular target region is near a skeletal landmark or joint. Based on the user input, the system may determine that the target region is bone and not vascular calcium. In some embodiments, the system may determine that a target region is near a skeletal landmark or joint based on, for example, preloaded anatomic mapping/locations stored in memory (e.g., memory 1204). Anatomic maps/locations stored in memory may be associated with corresponding positions of the TUS device relative to various different anatomic regions of a subject.

Bones are generally located beneath layers of muscle, fat, and/or tendons, whereas arterial vessels (and thus vascular calcium) are generally located at shallower depths (in relation to a skin surface) compared to that of bones. In some embodiments, hardware processor 1202 may be configured to determine a distance of a target region below a skin surface based on acoustic data received from one or more acoustic sensors. For example, the processor 1202 may be configured to determine the depth of the imaged region on the time taken for emitted acoustic waves to travel to the target region and return (e.g., reflect) to a detector (e.g., time of flight). Travel time may correspond to distance under the skin surface by the target region. For example, a long acoustic wave travel time may correspond to greater distance under a skin surface by the target region than does a short acoustic wave travel time, which may correspond to a lesser distance under the skin surface by the target region.

In some embodiments, the system may differentiate between vascular calcium vessel and hard tissue such as bone based on whether a determined distance (of the target region) under a skin surface satisfies a distance threshold, such as a predetermined distance threshold. The threshold can include limits such as lower and upper limits. In some embodiments, the threshold limit may be based on the extremity that comprises the target region, such as whether the target region is part of the thigh, calf, ankle, foot, etc. In some embodiments, the hardware processor 1202 may determine that a region of hard tissue is vascular calcium and not bone based on a determination that the distance under the skin surface of the targeted anatomic region satisfies the distance threshold. For example, hardware processor 1202 may determine that a region of hard tissue is vascular calcium and not bone based on a determination that the determined depth of the targeted anatomic region under the skin surface is less than a predetermined distance threshold value. In some examples, the hardware processor 1202 may determine that a region of hard tissue is bone and not vascular calcium based on a determination that the determined depth of the targeted anatomic region under the skin surface is at or greater than the predetermined distance threshold value.

Distance thresholds may be fixed or programmable. In some aspects, distance thresholds can be set and/or modified by the wearer/user utilizing any device described herein. In some aspects, distance thresholds can be set and/or modified by the wearer/user utilizing the TUS wearable device (e.g., device 500) or a non-wearable device via a device application (e.g., a smart watch application, a smartphone application, etc.) or a web-based application. In some embodiments, distance thresholds may be set based on the extremity being targeted for TUS treatment and/or imaging.

In some embodiments, the system may differentiate between regions of vascular calcium and other hard tissues (e.g., bone) based on various ultrasound detection considerations described herein. For example, in some embodiments, the hardware processor 1202 may be configured to arbitrate between various acoustic information such as detected/measured acoustic features (e.g., reflected acoustic energy, ratios of reflected acoustic energy, voltage levels, etc.), detected/measured blood flow and/or flow rate, determined anatomic characteristics (e.g., size, location, depth, etc.), other ultrasound imaging considerations, and/or combinations thereof, to determine whether a targeted anatomic region is vascular calcium. In some examples, the hardware processor 1202 may be configured to arbitrate between various ultrasound detection considerations according to multiple different thresholds, such as described herein. For example, in some embodiments, the hardware processor 1202 may determine that a targeted anatomic region is vascular calcium rather than bone based at least in part on a determined structural size of the hard tissue and detected blood flow in the same target region (such as blood flow above and/or below the vascular calcium).

In some embodiments, the TUS system may utilize one or more transducers (e.g., therapeutic transducers operating in a diagnostic mode, separate imaging transducers, etc.), and corresponding sensors, to perform one or more detection techniques, such as described herein. Advantageously, image-guided TUS may provide enhanced acoustic and/or visual information regarding a targeted anatomic region. For example, one or more transducers may emit acoustic waves directed toward the anatomic region. Corresponding sensors, such as acoustic sensors, may collect acoustic data based on acoustic waves detected after reflection by the targeted anatomic region for processing by hardware processor 1202. The hardware processor 1202 may determine various acoustic information such as one or more acoustic features (e.g., reflected energy, ratio of reflected energy, voltage levels, etc.), detected/measured blood flow and/or flow rate, and/or one or more determined anatomic characteristics (e.g., size, location, and/or depth of the target region) based on various acoustic data originating from the one or more acoustic sensors. Additionally, or alternatively, the system may image the targeted anatomic region using one or more transducers (such as arranged in an imaging array), and corresponding sensors may collect image data based on acoustic waves detected after reflection by the targeted anatomic region for processing by hardware processor 1202. Based on the image data, the hardware processor 1202 may generate and output to the user (e.g., via display 1214) a visualization of the targeted region.

Advantageously, the system may provide to the user a combination of acoustic information (based on the processed acoustic data) and visual information (based on the processed image data) that may include more detailed anatomic information than simply providing acoustic information or visual information alone. For example, in some embodiments, the system may provide to the user an “exact” ratio of reflected energy (such as based on the processed acoustic data) while also providing a visualization (such as based on the processed image data) of the targeted region. In some embodiments, the system may determine acoustic and visual information associated with acoustic waves emitted by a single transducer. In some embodiments, the system may determine acoustic and visual information associated with acoustic waves emitted by different transducers. For example, the system may determine acoustic information based on acoustic data associated with acoustic waves emitted by a single transducer (or an array of such transducers), and the system may determine visual information based on image data associated with acoustic waves emitted by another transducer (such as another array of transducers).

In response to a determination that the imaged anatomical region is not arterial vessel and/or vascular calcium, the system may selectively deactivate (e.g., turn off, prohibit from emitting acoustic waves) one or more transducers. For example, the hardware processor 1202 can be configured to deactivate one or more transducers 1210 and generate an indication to the wearer/user that the transducer(s)/array(s)/treatment device needs to be repositioned. Indicia may include visual, tactile/haptic, and/or auditory feedback. The hardware processor 1202 may generate the indication in a user interface of a display (e.g., display 1214). In some aspects, the hardware processor 1202 may be configured to generate instructions, such as in a user interface of the display, directing the wearer/user where to move the device (e.g., indicating a direction and/or distance to move the device).

In some embodiments, one or more transducers may be deactivated based on detection of high reflected acoustic power (e.g., reflected acoustic energy greater than a threshold) at those transducers, and remaining transducers, at which low reflected power is detected (e.g., reflected acoustic energy less than or at a threshold) may remain active (e.g., turned on, permitted to emit acoustic waves) to continue to provide delivery of therapeutic energy.

In response to a determination that the imaged anatomical region is arterial vessel and/or vascular calcium, the hardware processor 1202 can be configured to deactivate one or more imaging transducers and activate one or more therapeutic transducers for delivery of therapeutic energy to the targeted tissue site. In some embodiments, the hardware processor 1202 may be configured to generate an indication to the wearer/user that the imaged anatomical region is an arterial vessel and/or vascular calcium. For example, the hardware processor 1202 may cause to be displayed in a user interface of a display (e.g., display 1214) indicia of vascular calcium. In some examples, the hardware processor 1202 may cause to be generated in the user interface a rectangle (or any other shape) around the region of vascular calcium in the image displayed in the display. In some embodiments, the hardware processor 1202 may be configured to generate indications including visual, tactile/haptic, and/or auditory feedback.

In some embodiments, a patient-driven method of placement can be utilized by using a “PUSH” technique (Place Until Softness Heard). As such, ultrasound intensity can be converted (such as from reflective acoustic pressure/voltage) into audible sound, or other indicia. When the device (e.g., device 500) is positioned over soft tissues (e.g. muscle, arteries such as arteries having regions of vascular calcium), the audible sound may be quiet (e.g., low intensity, low frequency, etc.). When the device is positioned over hard tissue (e.g., bone), the audible sound may be loud (e.g., high intensity, high frequency, etc.). In some embodiments, the audible sound can be binary, such as a warning beep or other alarm if the device is placed over hard tissues, and no sound or a confirmatory pleasant tone and/or the like if the device is placed over soft tissues. In some embodiments, the indicia may include a visual signal (e.g., a change in brightness or color change on an LED light or display), a quantitative score on a display (e.g., display 1214), tactile/haptic feedback such as different degrees of vibration, or any other indicia that can indicate to the patient or provider that the device is positioned over soft tissues or hard tissues.

In some embodiments, imaging can serve as an intermittent (within days or weeks) confirmation of effects and mechanism of revascularization. As described hereinabove, Doppler imaging may be utilized to determine blood flow so as to evaluate revascularization of targeted arterial vessels. If, for example, a patient responded to TUS treatment, therapy can be continued; or if response to treatment was not optimal, a TUS parameter can be modified to improve response.

It could be useful in some cases to detect measures of blood flow and perfusion on the same platform/system that delivers the acoustic energy, or a different system. Light-based sensors that detect scattering (e.g., diffusion correlation spectroscopy or diffuse speckle contrast analysis techniques) or absorption as a function of hemoglobin concentrations in circulating blood can be incorporated. Thus, in some embodiments, an emitter such as an LED and a detector (e.g., sensor) can be placed in the ultrasound field, or proximal or distal to the site of ultrasound application, to determine acute (within minutes or hours) or chronic (within days or weeks) changes in perfusion as a sign of response and success of therapy. In some embodiments, direct or indirect improvements in perfusion may be assessed and quantitated by perfusion-specific magnetic resonance imaging, laser Doppler imaging, angiography (including CT, MR, and intra-arterial catheter angiography, and fluorescence microangiography), systematic ulcer/wound assessment, microbubble ultrasound perfusion imaging, ankle-brachial index, toe-brachial index or transcutaneous oximetry (TcPO₂).

The display 1214 can display user interfaces such as any of the example user interfaces, or aspects thereof, that are shown and/or described herein. The display 1214 can include an LED screen, an LCD screen, an OLED screen, a QLED screen, a plasma display screen, a quantum dot display, or the like. The display 1214 can be a colored display or a monochrome display. The display 1214 may be responsive to touch. For example, the display 1214 may comprise a touch screen such as a resistive touch screen, a capacitive touchscreen, an infrared touchscreen, a surface acoustic wave touchscreen, or the like. In some aspects, the display 1214 can display various information such as information based on data gathered from sensor(s) 1212. In some aspects, the displayed information may be based on data gathered from one or more components/devices of system 800, such as a wearable or nonwearable TUS device (e.g., device 500), an imaging device 818, and/or the like.

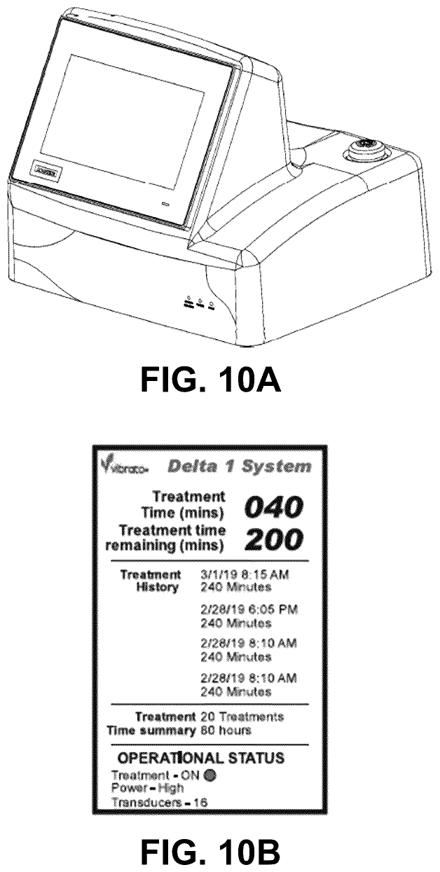

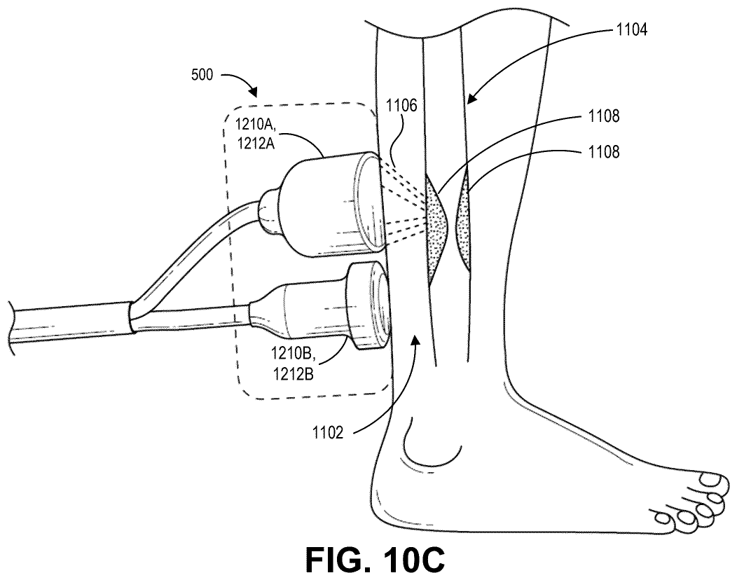

FIGS. 10A-10B illustrate an ultrasound generator and patient interface, respectively. FIG. 10A schematically illustrates an ultrasound generator configured for use with the TUS wearable device. The generator illustrated in the example of FIG. 10A may be implemented as, or include one or more features and/or functions of, power source 1208 described herein with reference to FIG. 9. FIG. 10B illustrates a display on the generator, which can be configured for patient interface and compliance tracking. The display illustrated in the example of FIG. 10B may be implemented as, or include one or more features and/or functions of, display 1214 described herein with reference to FIG. 9. In some embodiments, the display can be, alternatively or in addition, on a separate computing device such as a remote smartphone, tablet, PC, or the like and configured to communicate (e.g., wired, wire-like, wirelessly) with the generator. In some embodiments, each generator unit may be configured to track the treatment time, date, and duration to verify use. The generator unit can include continuous electronic monitoring of the power being delivered to each of the transducers to ensure the desired acoustic dosage is delivered for each clinical encounter and provide an additional margin of safety. Reflected power (which can indicate poor coupling between transducer and skin) from each transducer can also be monitored, and power to one or more transducers can be turned off to minimize over-heating or transducer damage. Further design features of the wearable device and/or generator system can include any number of the following:
connection ports (e.g., one port for each strip of transducers)
Integrated matching network and patient isolation
single board PC with graphic display
hidden USB port for data download and software upgrade uploads
treatment data file log storage
transducer parameter logs

FIG. 10C illustrates an example embodiment of a TUS device 500, such as described herein. The TUS device 500 may be coupled to the lower extremities of a patient. In the example embodiment illustrated in FIG. 10C, the TUS device is coupled to the patient at the level of the calf. However, this is not intended to be limiting. The TUS device 500 may be coupled to any other portion of the lower extremities (e.g., the thigh, ankle, foot, etc.), or any other extremities of the patient. The TUS device 500 can include a plurality of transducers 1210. Transducers 1210 can include therapeutic transducers and/or imaging transducers, such as described herein. In some embodiments, the TUS device 500 can include an array of transducers (e.g., array 1000 such as shown and/or described in FIG. 5). The TUS device 500 can include a plurality of sensors 1212. Sensors 1212 can include acoustic sensors and/or any other sensor described herein, such as are suitable for TUS treatment and/or imaging. The TUS device 500 may be configured as a wearable device. In some embodiments, the TUS device 500 may be configured as a handheld device that is movable to multiple different anatomic regions of a patient and/or movable to multiple different portions of a single anatomic region. For example, the TUS device 500 can be implemented as, or include one or more features and/or functions of, device 818 such as shown and/or described in FIG. 8. The TUS device 500 may be in wired, wire-like, or wireless communication with transducers 1210 and/or sensors 1212 via a network, such as network 808 shown and/or described in FIG. 8. In some embodiments, the TUS device 500 can be in wired, wire-like, or wireless communication with an ultrasound generator (FIG. 10A) via, for example, network 808. In some embodiments, a user may control one or more functions of the TUS wearable device 500 via interacting with a display (FIG. 10B) of the ultrasound generator.

In the example embodiment illustrated in FIG. 10C, the TUS device 500 includes a therapeutic transducer 1210A and an imaging transducer 1210B. The transducer 1210A can be aligned with the imaging transducer 1210B, such as vertically or horizontally. As illustrated in FIG. 10C, the therapeutic transducer 1210A is positioned above the imaging transducer 1210B; however, this is not intended to be limiting. Transducers 1210A and 1210B may be arranged in any configuration suitable for image-guided TUS treatment. Advantageously, combined use of therapeutic and imaging transducers can be beneficial to a patient to help ensure that therapeutic energy is directed to regions of vascular calcium and not to bone or other unwanted areas. Moreover, image-guided TUS treatment can help ensure that treatment remains safe, such as by monitoring for thrombosis and/or damage to vessels and/or other surrounding issue. In some embodiments, ultrasound imaging can be used to evaluate the effectiveness of TUS treatment, such as by identifying the location of cavitation and/or identifying indications of increased blood flow through targeted arterial vessels, such as described herein.

TUS device 500 may be positioned on the skin surface 1102 such that transducers 1210A, 1210B are aligned or substantially aligned with an underlying arterial vessel, such as arterial vessel 1104. It is to be understood that arterial vessel 1104 has been included for purposes of illustration, and may not accurately depict the anatomy of a patient, such as the anatomy of the calf region of the patient. In some embodiments, the TUS device 500 can include a plurality of therapeutic transducers 1210A and/or a plurality of imaging transducers 1210B, such as arranged in a transducer array. The TUS device 500 may be positioned on the skin surface 1102 such that the transducer array may is aligned or substantially aligned with an underlying arterial vessel, such as arterial vessel 1104.

Transducer 1210A can be configured to emit acoustic waves to deliver therapeutic energy 1106 at a region of vascular calcium 1108, for example, at the level of the calf. The TUS device 500 can cause the transducer 1210A to focus an acoustic field 1106 at or near a region of vascular calcium 1108 to generate cavitation microbubbles, such as a cavitation cloud. In some embodiments, the device 500 can cause the transducer 1210A to focus an acoustic field 1106 at or near the region of vascular calcium 1108 to generate shear stress at the level of the calcium. Localized shockwaves from bursting microbubbles may condition the region of vascular calcium 1108 such that the afflicted arterial vessel 1104 is revascularized (e.g., increased blood flow through the vessel). In some embodiments, for example for large arterial vessels, cavitation may condition the vascular calcium sufficient for subsequent PAD interventions such as invasive interventions, including catheter-based revascularization. Emitted acoustic waves may generate cavitation microbubbles at various locations of the targeted vessel 1104, such as at the front surface of the vessel, the middle of the calcium, and/or or the back surface of the vessel. In some embodiments, shear stress imparted by the emitted acoustic waves 1106 themselves may condition the vascular calcium 1108 to promote revascularization, such as described herein. In some embodiments, TUS treatment may include combinations of the aforementioned treatment mechanisms.

Transducer 1210B can be configured to emit acoustic waves for ultrasound imaging, for example, at the level of the calf. Transducer 1210B can be utilized in combination with any available type of ultrasound imaging technique, such as any of the ultrasound imaging techniques described herein. The TUS device can operate transducer 1210B and transducer 1210A in an alternating scheme. For example, during imaging of the calf region (or any other anatomic region), the device 500 can activate imaging transducer 1210B and deactivate therapeutic transducer 1210A. Upon detection of vascular calcium 1108, the device 500 can activate therapeutic transducer 1210A and deactivate imaging transducer 1210B. After directing therapeutic energy to the region of vascular calcium 1108, the TUS device 500 can deactivate the therapeutic transducer 1210A and activate the imaging transducer 1210B to proceed with imaging/detection of another region of vascular calcium, for example, as the TUS device 500 scans the target vessel 1104 (such as via mechanical and/or electronic beam steering). Although the example embodiment illustrated in FIG. 10C illustrates two separate transducers, this is not intended to be limiting. In some embodiments, a single transducer can be configured to emit acoustic waves effective for delivery of therapeutic energy at a first time and acoustic waves effective for ultrasound imaging at a second time (e.g., such as when in a diagnostic mode).

The TUS device 500 can cause transducers 1210A and/or 1210B to emit acoustic waves at various frequencies, pulse durations, pulse frequencies, acoustic intensities, and/or any other TUS parameter discussed herein, such as is suitable for TUS treatment and/or ultrasound imaging. In some embodiments, the TUS device 500 may operate transducers 1210A and/or 1210B at various different duty cycles (such as any duty cycle described herein) depending on treatment needs. For example, the device 500 may be configured as a closed-loop system and adjust TUS parameters based on, for example, acoustic data originating from one or more sensors. In some examples, the device 500 may adjust TUS parameters based on received user input via, for example, a user interface and/or interface console.

In some embodiments, the TUS device 500 can adjust the focal depth of the acoustic field 1106 to target various different regions of vascular calcium 1108. In some aspects, transducers 1210A and/or 1210B may be arranged in a particular geometric arrangement (e.g., parabolic arrangement) to implement geometric focusing, as described herein. In some aspects, the device 500 can control phase delays and/or time delays in control signals sent to transducers 1210A and/or 1210B to implement electronic focusing, as described herein.

In some embodiments, the TUS device 500 can be configured for mechanical and/or electronic beam steering. For example, transducers 1210A and/or 1210B may be coupled to mechanical actuators or tilting mechanisms, such as described herein, that adjust the position of the transducer such that the transducer can emit acoustic waves in multiple different directions. In some examples, as described herein, the device 500 may implement electric switching and select different transducers (e.g., subsets of the transduces), such as in a sequence, to emit acoustic waves from transducers disposed at different portions of the device (e.g., from different portions of a transducer array) such that the acoustic waves are directed to different regions of a target vessel.

The TUS device 500 can include one or more sensors. In the example embodiment illustrated in FIG. 10C, the device includes sensors 1212A and 1212B. Sensors 1212A and/or 1212B may be integrated into, housed by, or connected to the device 500. In some embodiments, sensors 1212A and/or 1212B may be integrated into, housed by, or connected to transducers 1210A and/or 1210B, respectively. In some embodiments, sensors 1212A and/or 1212B may be separate from transducers 1210A and/or 1210B, and/or separate from the TUS device 500. The sensors 1212A and/or 1212B can be in wired, wire-like, or wireless communication with the device 500.

Sensors 1212A and/or 1212B can be configured to obtain acoustic data and or other data, such as physiological data or image data, from the patient. Acoustic data can include any of the data described herein. The sensors 1212A and/or 1212B may generate signals indicative of acoustic information and/or TUS parameters, such as described herein. In some embodiments, the sensors 1212A and/or 1212B may generate signals indicative of image information and/or physiological information, such as described herein. The sensors 1212A and/or 1212B may transmit such data to other components of the TUS device 500 (e.g., hardware processor(s) 1202 shown and/or described in FIG. 9) for processing. In some embodiments, sensor 1212A and sensor 1212B can be in communication with each other. In some embodiments, the TUS device 500 can include a single sensor capable of obtaining multiple different types of acoustic, image, and/or physiological data. In some embodiments, the TUS device 500 can include a plurality of sensors, such as arranged in a sensor array suitable for collecting acoustic, image, and/or physiological data during image-guided TUS treatment.

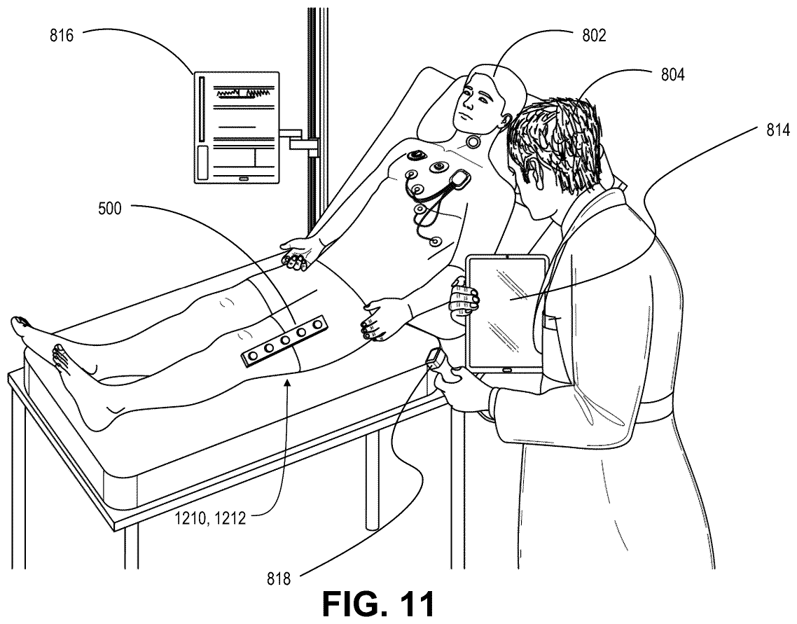

FIG. 11 illustrates an example implementation of TUS system 800. A TUS wearable device 500 can be coupled to the lower extremities (e.g., at the level of the thigh) of the patient 802. The wearable device can include one or more transducers 1210 as described herein and/or transducer arrays (e.g., array 1000 described with reference to FIG. 5). In some embodiments, transducers 1210 can include therapeutic transducers and/or imaging transducers as described herein. One or more transducers and/or transducer arrays, in some embodiments, may be coupled to other extremities of the patient 802. The wearable device 500 can include one or more sensors 1212. Sensors 1212 can include acoustic sensors and/or any other sensor described herein. In some embodiments, one or more sensors may be secured to other extremities of the patient. The wearable device 500 may be in wired, wire-like, or wireless communication with transducers 1210 and/or sensors 1212 via, for example, network 808.

One or more transducers 1210 can be configured to emit acoustic waves to deliver therapeutic energy at a region of vascular calcium, for example, at the level of the thigh. In some embodiments, the device 500 may cause one or more transducers to focus an acoustic field at or near a region of vascular calcium to generate cavitation microbubbles (e.g., a cavitation cloud) and/or shear stress at the level of the calcium. Localized shockwaves from bursting microbubbles may condition the region of vascular calcium such that the afflicted arterial vessel is revascularized (e.g., increased blood flow through the vessel). In some embodiments, for example for large arterial vessels, cavitation may condition the vascular calcium sufficient for subsequent PAD interventions such as invasive interventions, including catheter-based revascularization. Emitted acoustic waves may generate cavitation microbubbles at various locations of the targeted vessel, such as at the front surface of the vessel, the middle of the calcium, and/or or the back surface of the vessel. In some embodiments, shear stress imparted by the emitted acoustic waves themselves may condition the vascular calcium to promote revascularization, such as described herein. In some embodiments, TUS treatment may include combinations of the aforementioned treatment mechanisms.

One or more transducers 1210 can be configured to emit acoustic waves for ultrasound imaging, for example, at the level of the thigh. As described herein, image-guided TUS treatment can be beneficial to the patient to help ensure that therapeutic energy is directed to regions of vascular calcium and not to bone or other unwanted areas. Moreover, image-guided TUS treatment can help ensure that treatment remains safe, such as by monitoring for thrombosis and/or damage to vessels and/or other surrounding issue. In some embodiments, ultrasound imaging can be used to evaluate the effectiveness of TUS treatment, such as by identifying the location of cavitation and/or identifying indications of increased blood flow through targeted arterial vessels, such as described herein.

The wearable device 500 can operate therapeutic and imaging transducers in an alternating scheme. For example, during imaging, the device can activate imaging transducers and deactivate therapeutic transducers. Upon detection of vascular calcium, the device can activate therapeutic transducers and deactivate imaging transducers. In some aspects, a single transducer can be configured to emit acoustic waves effective for delivery of therapeutic energy at a first time and acoustic waves effective for imaging at a second time. In some aspects, different transducers (e.g., different subsets of transducers, different transducer arrays, etc.) may be used for TUS treatment and for imaging. After treating the region of vascular calcium, the wearable device 500 can deactivate the therapeutic transducers and activate the imaging transducers to proceed with imaging/detection of another region of vascular calcium, for example, as the wearable device 500 (or imaging device 818) scans the target vessel (such as via mechanical and/or electronic beam steering).

The wearable device 500 can cause one or more transducers to emit acoustic waves at various frequencies, pulse durations, pulse frequencies, acoustic intensities, and/or any other TUS parameter discussed herein. In some embodiments, the wearable device may operate one or more transducers at various different duty cycles (such as any duty cycle described herein) depending on treatment needs. For example, the device may be configured as a closed-loop system and adjust TUS parameters based on, for example, received acoustic data. In some examples, the device may adjust TUS parameters based on received user input via, for example, a user interface and/or interface console.

In some embodiments, the wearable device 500 can adjust the focal depth of the acoustic field to target various different regions of vascular calcium. In some aspects, the device can include one or more transducers positioned in a particular geometric arrangement (e.g., parabolic arrangement) to implement geometric focusing, as described herein. In some aspects, the device can control phase delays and/or time delays in control signals sent to one or more transducers to implement electronic focusing, as described herein.

In some embodiments, the wearable device 500 can be configured for mechanical and/or electronic beam steering. For example, one or more transducers may be coupled to mechanical actuators or tilting mechanisms, such as described herein, that adjust the position of the transducer such that the transducer emits acoustic waves in a different direction. In some examples, as described herein, the device may implement electric switching and select different transducers (e.g., subsets of the transduces), such as in a sequence, to emit acoustic waves from transducers disposed at different portions of the device (e.g., from different portions of a transducer array) such that the acoustic waves are directed to different regions of a target vessel.

One or more sensors 1212 can be configured to obtain acoustic data and or other data, such as physiological data, from the patient. Acoustic data can include any of the data described herein. The sensors may generate signals indicative of acoustic/TUS parameters and/or other parameters described herein. The sensors may transmit such data to other components of the TUS system, such as the wearable device 500 for processing. In some embodiments, sensors 1212 can be in communication with each other.

The imaging device 818 may be a handheld device that includes one or more transducers configured to emit acoustic waves effective for ultrasound imaging. For example, the imaging device can be implemented as an ultrasound imaging probe. The imaging device 818 may be in wired, wire-like, or wireless communication with the TUS wearable device 500 and/or other components of the system 800, such as one or more transducers/sensors.

In the example of FIG. 11, the imaging device is a separate device from the wearable device. However, this is not intended to be limiting. The imaging device 818 may be coupled to the same location as the TUS wearable device 500, and/or may include one or more features and/or functions of the device 500. For example, the imaging device 818 and device 500 may be integrated as a single device for image-guided TUS treatment. In some examples, the imaging device 818 can include imaging transducers and therapeutic transducers, and may be configured to operate the transducers in an alternating manner, such as described hereinabove. The imaging device 818 can be used to position one or more transducers/transducer arrays, direct an acoustic field in a certain direction and focus the acoustic field at a certain depth, such as described herein. The imaging device 818 can be implemented according to an open-loop or closed-loop configuration, such as for continuous monitoring of acoustic data/image data during a treatment period.

The monitoring hub 816 and the tablet 814 can be in wired, wire-like, or wireless communication with one or more devices/components of system 800, such as the TUS wearable device 500, the imaging device 818, and/or one or more transducers/sensors. The monitoring hub 816 and tablet 814 can be in communication with each other. The monitoring hub/tablet can receive data (e.g., physiological data, acoustic data, image data) from one or more components, such as sensors 1212 (e.g., in real-time as that data is generated by the one or more sensors 1212). The monitoring hub/tablet can display indicia of the data and information relating thereto on a display of the monitoring hub/tablet (e.g., display 1214). In the example of FIG. 11, the monitoring hub 816 is shown being fixed in a location (e.g., mounted to a wall). The monitoring hub 816 may be removably mounted to the wall and may be portable or mobile (e.g., not fixed to a particular location)

The monitoring hub 816 and/or tablet 814 can generate or output alarms and/or notifications. An alarm or notification can comprise visual, auditory, and/or tactile feedback. For example, monitoring hub 816 can output a visual indication on display screen indicative of a particular physiological status of the patient 802 based on physiological data originating from the sensors 1212. In some examples, the monitoring hub 816 can output a visual indication on a display screen indicative of the TUS treatment status of the patient 802 based on one or more of acoustic data, physiological data, and/or image data originating from one or more devices/components of system 800, such as TUS wearable device 500, imaging device 818, sensors 1212, and/or the like. In some examples, the hub 816 can output a visual indication on a display screen indicative of the operation status of various devices/components of system 800, such as whether one or more transducers are correctly positioned, whether one or more transducers are delivering therapeutic energy (and the associated TUS parameters), whether one or more sensors are disconnected, and/or the like. In some embodiments, monitoring hub 816 can output an audio signal or tactile/haptic feedback indicative of the statuses discussed hereinabove.

In some embodiments, monitoring hub 816 and/or tablet 814 can generate instructions to a user (e.g., subject 802, user 804) to position the transducers in a certain location and/or to move the transducers in a certain direction and/or by a certain distance along a skin surface. In some embodiments, the instructions can include visual, auditory, and/or tactile/haptic feedback. Feedback can include indications that transducers are correctly or incorrectly positioned. In some embodiments, the monitoring hub 816 and/or tablet 814 may continually update the instructions until the transducers have been properly positioned. For example, acoustic/image parameters may be continually recalculated (e.g., in an open or closed loop) and instructions generated based on the most recent acoustic/image data.

Various other computing device may be present in the room illustrated in the example of FIG. 11, such as mobile communication devices (e.g., device 806), desktops (e.g., device 810), laptops (e.g., device 812), and/or the like. The features and/or functions described herein in relation to monitoring hub 816 and/or tablet 814 may be integrated into and/or implemented by one or more devices/components of TUS system 800, such as the wearable device 500, the imaging device 818, a mobile communication device (e.g., device 806), desktops/laptops (e.g., devices 810, 812), and/or the like.

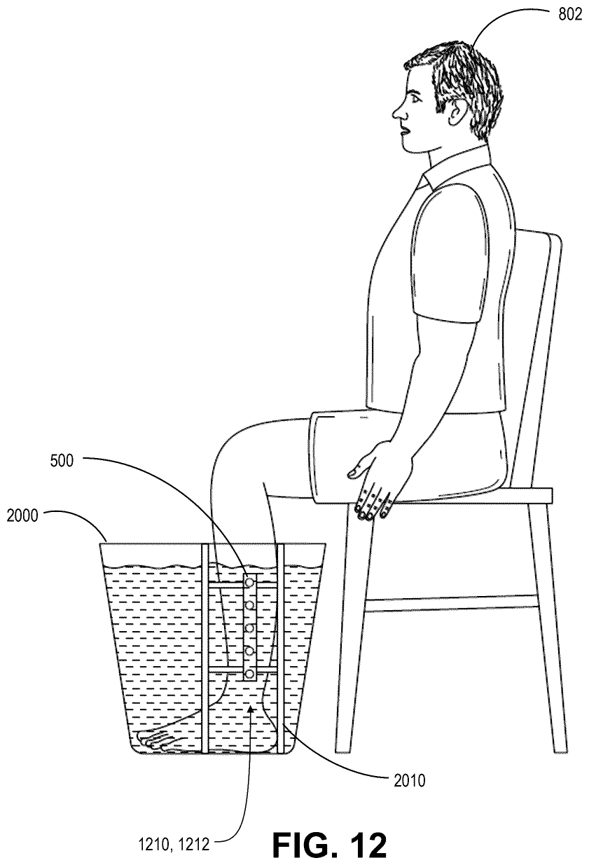

FIG. 12 illustrates an example implementation of system 800. In the example of FIG. 12, the patient 802 positions their foot in a water bath 2000. A TUS wearable device 500, including one or more transducers (e.g., transducers 1210) and/or sensors (e.g., sensors 1212), can be coupled to the lower extremities (e.g., at the level of the calf) of the patient 802. The wearable device 500 may be submerged in the water bath 2000.

In some embodiments, the wearable device 500 may be integrated into, housed by, or connected to a movement device 2010 configured to facilitate mechanical translation along and/or rotation about an anatomical region (such as the calf). For example, the movement device 2010 can include one or more motors for motorized translation/rotation of the wearable device 500. The movement device 2010 may be disposed within the water bath 2000 and may extend along at least a portion of the lower extremities of the patient 802 such that one or more transducers and/or sensors are positioned proximate or at the skin surface for effective TUS treatment and/or ultrasound imaging.

The movement device 2010 may move the wearable device 500 along a path on the skin surface that is aligned (or substantially aligned) with an underlying arterial vessel. The movement device 2010 may be configured to move the wearable device 500 to different anatomical regions of the lower extremities, including to the thigh, foot, and/or ankle. Within a single anatomical segment, the movement device 2010 can be configured to rotate the wearable device 500 about the anatomical segment to target the anterior, posterior, medial, or lateral side of, for example, the calf based on desired target vessel.

Although the example of FIG. 12 illustrates a movement device extending a long a portion of the patient's calf, this is not intended to be limiting. Such a device may be utilized to facilitate mechanical translation along and/or rotation about any anatomical region of the lower extremities, such as the thigh, calf, ankle, and/or foot.

In some cases, based on time duration of TUS treatment, it may be advantageous to implement a water bath. In some cases, a water bath can help provide a temperature-controlled environment for precise temperature regulation during therapy (e.g., preventing transducers from getting too hot). Moreover, when transducers/sensors are coupled to a body part immersed in water, the water can help to reduce (or minimize) movement of the targeted body part and stabilize the transducers/sensors. This can help improve accuracy of measurements, especially during periods of long-term monitoring/TUS treatment. Consequently, utilizing a water bath may permit use of transducers/sensors having simple designs, which can reduce the cost of treatment. Additionally, use of a water bath can reduce (or minimize) issues associated with operator fatigue (e.g., of a handheld device) during long-term treatment. For example, the water bath may be used in combination with image-guided TUS, such as described herein. In some examples, an imaging device (e.g., imaging device 818) may be integrated into the wearable device 500 and/or integrated into, housed by, or connected to the movement device 2010 such that the imaging device can move in tandem with the TUS wearable device.

Water baths may also provide advantageous therapeutic effects for the patient 802, such as thermal therapeutic effects that may be used in combination with TUS treatment. For example, warm water can help dilate blood vessels and relax surrounding tissue for increased blood flow in a target area. The buoyancy of the water can help reduce (or minimize) pressure on the treated skin surface, making it more comfortable for the patient to undergo prolonged TUS treatment.

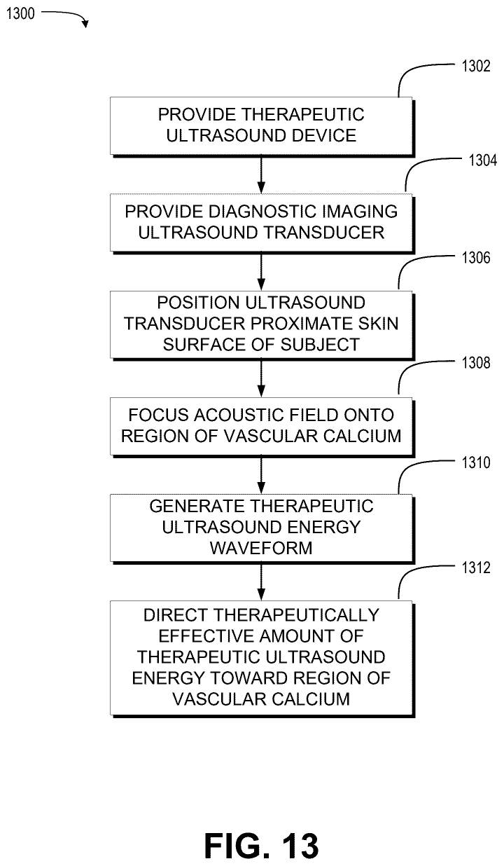

FIG. 13 illustrates an example process of performing TUS treatment using a TUS wearable device, such as TUS wearable device 500. In some aspects, the process 1300, or portions thereof, can be performed by the wearable device 500, the imaging device 818, the computing device 1200, or any other computing device described herein. In some aspects, the process 1300, or portions thereof, may be performed by a hardware processor, such as processor 1202. In some aspects, the process 1300, or portions thereof, can be performed by a user of the device, such as a medical practitioner.

At block 1302, a therapeutic ultrasound device can be provided for TUS treatment. For example, the user may provide the TUS medical device. The TUS device can be configured to generate a therapeutic ultrasound acoustic field. The device can include the wearable device 500, the imaging device 818, the computing device 1200, one or more transducers 106, 900, 901, 1210, transducer arrays 1000, and/or any other computing device configured for TUS treatment as described herein. In some aspects, at block 1302, the device can be a wearable device or a non-wearable (e.g., handheld) device.

At block 1304, a diagnostic imaging ultrasound transducer can be provided. For example, the user may provide the ultrasound transducer. The diagnostic ultrasound transducer can be coupled to or used in conjunction with the TUS device. In some aspects, the diagnostic imaging ultrasound transducer can be configured to display a location of a therapeutic ultrasound acoustic field and superimpose indicia of the TUS filed on the diagnostic ultrasound image. The transducer can include any of transducers 106, 900, 901, 1210 configured for ultrasound imaging (e.g., configured to emit acoustic waves effective for imaging).

At block 1306, an ultrasound transducer (e.g., TUS transducer and/or imaging transducer) can be positioned proximate a skin surface of a subject. For example, the user may position the ultrasound transducer proximate the skin surface of the subject. In some examples, at block 1306, the transducer may be positioned at or near the skin surface. At block 1306, the transducer may be coupled to the skin surface, such as according to any of the mechanical and/or chemical coupling means described herein. For example, at block 1306, the transducer may be coupled to the skin surface via a sleeve, straps, bands, clips, clamps, hook-and-loop fasteners, suction, adhesives (e.g., gels, skin-friendly glue, tape, cream, etc.), and the like. In some aspects, at block 1306, the ultrasound transducer may be integrated into, housed by, or connected to a movement device that positions the transducer proximate the skin surface for movement along the skin surface (e.g., translation along/rotation about the skin surface). The transducer can include any of transducers 106, 900, 901, 1210 configured for ultrasound therapy and/or ultrasound imaging (e.g., configured to emit acoustic waves effective for TUS treatment of vascular calcium and/or effective for imaging).

At block 1308, an acoustic field of the TUS transducer can be focused onto a region of vascular calcium within a vessel of the subject below the skin surface. For example, the user and/or TUS device may cause the TUS transducer to focus an acoustic field onto the region of vascular calcium. Focusing an acoustic field on a region of vascular calcium may include causing emitted acoustic waves to constructively interfere and/or destructively interfere at a certain region of vascular calcium to, for example, increase the intensity of acoustic field. Focusing an acoustic field may include focusing the acoustic field at various different focal depths to target various different regions of vascular calcium.

In some embodiments, at block 1308, the TUS device can cause the acoustic field to focus onto the region of vascular calcium via geometric focusing. For example, one or more TUS transducers may be arranged in a curvilinear or parabolic shape to permit geometric focusing. Such an arrangement may cause acoustic waves emitted by the transducer to converge (e.g., focus) at a specific point, such as the region of vascular calcium. For example, a parabolic arrangement of transducers can focus therapeutic energy at a focus of the parabola.

In some embodiments, at block 1308, the TUS device can cause the acoustic field to focus onto the region of vascular calcium via electronic focusing. For example, the TUS device may cause to be adjusted the phases of signals sent to the transducers to create a virtual curvature in the emitted acoustic wavefront (e.g., based on constructive and/or destructive interference of acoustic waves) such that the acoustic field can be focused to the region of vascular calcium. In some examples, at block 1308, the TUS device may cause to be introduced time delays in sending signals to the transducers to produce a virtual curvature in the emitted acoustic wavefront (e.g., based on constructive and/or destructive interference of acoustic waves) such that the acoustic field can be focused to the region of vascular calcium.

At block 1310, a therapeutic ultrasound energy waveform can be generated. At block 1310, the user and/or TUS device can cause the ultrasound transducer to emit a therapeutic ultrasound energy waveform having a therapeutically effective amount of therapeutic ultrasound energy. For example, the TUS device may be configured to cause the transducer to emit an acoustic wave at a pulse repetition frequency and having an acoustic frequency, a pulse duration, a peak negative pressure, and/or an acoustic intensity sufficient to promote revascularization of the targeted arterial vessel. At block 1310, a therapeutically effective amount of therapeutic ultrasound energy can generate cavitation microbubbles that, upon collapse, impart localized shockwaves at the level of the calcium sufficient to revascularize the afflicted arterial vessel and/or condition the vascular calcium sufficient for subsequent PAD intervention, such as catheter-based revascularization. In some embodiments, at block 1310, a therapeutically effective amount of therapeutic ultrasound energy can generate shear stress sufficient to revascularize the afflicted arterial vessel and/or condition the vascular calcium sufficient for subsequent PAD intervention, such as catheter-based revascularization.

At block 1310, conditioning vascular calcium can include reducing the hardness and/or rigidity of the vascular calcium (e.g., softening the vascular calcium). Conditioning vascular calcium may include disrupting/fracturing (e.g., inducing microfractures in the vascular calcium) via acoustic shockwaves. Conditioning vascular calcium may improve (e.g., increase) the flexibility and/or elasticity of the afflicted arterial vessel such that the vessel has an improved response to pulsatile blood flow through the vessel and/or is more amenable to invasive PAD interventions.

At block 1312, a therapeutically effective amount of TUS energy can be directed toward the region of vascular calcium. For example, the user and/or TUS device may cause the ultrasound transducer to emit acoustic waves having sufficient TUS energy to effectively treat a region of vascular calcium. At block 1312, in some embodiments, the user and/or TUS device can cause a therapeutically effective amount of TUS energy to be directed toward the region of vascular calcium to stimulate cavitation and shear stress and to remove or soften a calcium deposit within the region of vascular calcium. Directing TUS energy toward a certain direction can be implemented via beam steering. Beam steering can include directing the main lobe of the emitted acoustic field in a specific direction.

At block 1312, in some aspects, the TUS device may direct acoustic waves toward the region of vascular calcium via mechanical beam steering. For example, one or more ultrasound transducers may be configured to emit an acoustic wave according to an end fire or broadside acoustic radiation pattern. The transducers may be mechanically moved (e.g., mechanical translation and/or rotation) such that the main lobe of the emitted acoustic field moves to target the region of vascular calcium. In some embodiments, the ultrasound transducer may be connected to an actuator (e.g., linear actuator) or tilting mechanism configured to adjust the position of the transducer to adjust the direction of the emitted acoustic waves.

In some aspects, at block 1312, the TUS device may direct acoustic waves toward the region of vascular calcium via electronic beam steering. For example, the TUS device may implement electric switching and select different transducer elements to direct emitted acoustic waves to a region of vascular calcium. In some examples, the TUS device may activate (e.g., turn on, cause to emit acoustic waves) a first transducer (or first subset of transducers) disposed at a first position of the skin surface. The first transducer may direct emitted acoustic waves to a first corresponding region of vascular calcium underlying the first position of the skin surface. After the TUS device treats the first region of vascular calcium, the TUS device may activate a second transducer (or second subset of transducers) disposed at a second position of the skin surface. The second transducer may direct emitted acoustic waves to a second corresponding region of vascular calcium underling the second position of the skin surface. In some aspects, the TUS device may activate one or more transducers and deactivate (e.g., turn off, prohibit from emitting acoustic waves) one or more transducers. For example, the TUS device may cause the first subset of transducers to emit acoustic waves and prohibit the second subset of transducers from emitting acoustic waves. The first and second subsets of transducers may include entirely different transducers or may include one or more of the same transducers.

### Additional Example Implementations and Details

In some embodiments, systems and methods as disclosed herein can treat or prevent PAD, renal artery stenosis, carotid stenosis, vertebro-basilar insufficiency, brachial stenosis, axillary stenosis, or atherosclerosis or stenosis or other disease of any other vessel (including those described and/or illustrated herein) or body structure, and related vascular diseases such as atrial fibrillation or other arrhythmias, congestive heart failure (including ischemic cardiomyopathy). In some embodiments, systems and methods can be used to treat Alzheimer's or vascular dementia, and/or TIAs or ischemic stroke (e.g., with an ultrasound-based external cap or a catheter-based intravascular device for example). An implantable embodiment could be used to treat or prevent congestive heart failure (including ischemic cardiomyopathy) or related ischemic cardiovascular diseases such as atrial fibrillation, ventricular tachycardia or ventricular fibrillation, or other arrhythmias. In some embodiments, at least one transducer is implanted in the body adjacent to the target tissue of interest, and acoustic energy applied to the diseased or ischemic tissue (e.g., myocardium) to improve function and prevent or treat disease. The implantable transducer system can be powered with an implantable battery, or powered inductively via an external power supply. Wearable or implanted energy-emitting (e.g., ultrasound) components can also be utilized to treat other organs or other anatomical sites of interest to promote revascularization, such as the brain, spinal cord, lungs, GI tract (e.g., liver, spleen, pancreas, colon), kidneys, or other sites. In some embodiments, one or both upper extremities (e.g., shoulder, arm, forearm, wrist, or hand) can be treated using a cuff, sleeve, glove, or other form factor device.

The pathophysiology of diabetic foot ulcers is multifactorial, but, in part, are due to micro- and macro-vascular ischemia. Embodiments of this device may also be used for the clinical application of diabetic ulcer healing.

Some embodiments may be used to treat acute limb ischemia, hypoperfusion due to trauma and/or restless leg syndrome, which can have pathophysiologic mechanisms of vascular dysfunction and ischemia. Some embodiments can be used to treat muscular atrophy, enhance exercise capacity, increase muscle growth, and/or shorten recovery time from injury, trauma, or heavy exertion for example.

“Treatment” is a broad term having its ordinary meaning. In some cases, treatment can mean at least an amelioration of the symptoms associated with the pathological condition afflicting the host, where amelioration is used in a broad sense to refer to at least a reduction in the magnitude of a parameter, e.g., symptom, associated with the pathological condition being treated, such as ischemia. As such, treatment includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition. For example, treatment of PAD can result in reduction of claudication including rest-induced ischemic pain and/or exercise-induced ischemic pain, improvement in the ankle-brachial or toe-brachial index or other measurement of perfusion (including radiographic), transcutaneous oxygen pressures, duplex peak systolic velocity or velocity ratios, ankle or toe pressures, healing of ulcers, infections, or other wounds, prevention of gangrene, or other parameters.

Systems and methods can also be used to treat ischemic injury as a result of trauma, battlefield injury, compartment syndrome, or even to enhance angiogenesis of tissues, organs, or even limbs after surgery or transplantation. In some embodiments, systems and methods as disclosed herein can be used to treat deep venous thrombosis (DVTs), such as with a wearable device as described elsewhere herein. Systems and methods could also potentially be utilized to treat pulmonary embolism or pulmonary hypertension (e.g., with a thoracic vest targeting the lungs, pulmonary arteries, pulmonary veins, and/or bronchial arteries for example) to aid revascularization directed toward the lung vasculature, and/or offloading strain of the right heart. In some embodiments, systems and methods as disclosed herein can be utilized to treat or prevent preeclampsia or eclampsia by focusing therapeutic energy toward the placenta using parameters as described herein. Not to be limited by theory, while not well understood, preeclampsia and eclampsia can be associated with poorly developed uterine placental spiral arterioles (which decrease uteroplacental blood flow during late pregnancy), a genetic abnormality on chromosome 13, immunologic abnormalities, and placental ischemia or infarction. Diffuse or multifocal vasospasm in the placenta can result in maternal ischemia, eventually damaging multiple organs, particularly the brain, kidneys, and liver. Factors that may contribute to vasospasm include decreased prostacyclin (an endothelium-derived vasodilator), increased endothelin (an endothelium-derived vasoconstrictor), and increased soluble Flt-1 (a circulating receptor for vascular endothelial growth factor). Systems and methods (e.g., an abdominal binder, sleeve, or other wearable or other form factor) can preferentially promote placental revascularization thereby preventing or reversing the pathophysiology of placental vascular insufficiency in disorders such as pre-eclampsia and eclampsia. Treatment or prevention of ulcers may also be used for diabetic foot ulcers, with or without concomitant PAD, with similar wearable ultrasound methods and systems inducing revascularization. Treatment of renal artery stenosis, acute or chronic renal failure can manifest as improved BUN, creatinine, GFR, blood pressure, plasma renin, angiotensinogen, angiotensin I, angiotensin II, ACE, or other parameters. Treatment of carotid artery stenosis can manifest as reduced transient ischemic attacks (TIA) or stroke.

In some embodiments, the system can also include a diagnostic component for measuring perfusion at the anatomical location being treated, including a Doppler ultrasound perfusion measuring device or an optical perfusion measuring device, e.g., diffuse correlation spectroscopy or diffuse speckle contrast analysis in order to provide qualitative and/or quantitative measures of blood flow prior to, during, and/or after treatment sessions, which can be output to a display (in real-time in some cases). In some embodiments, the perfusion or other data can be utilized as a closed-loop feedback parameter to control or adjust therapy. In some embodiments and not to be limited by theory, Doppler or other blood flow measurements can be used to detect vasodilation using the same transducer or array, and use an acute blood flow increase above a predetermined threshold (e.g. about or at least about 5%, 10%, 15%, 20%, 25%, 30%, 35%, 40%, 45%, 50%, or more) as a proxy for ultimate revascularization effects. Above parameters (peak negative pressure, frequency, continuous or pulsed wave, duration, etc. can be thus customized/personalized to dial-in tailored acoustic parameters for a given patient and anatomic site, since local factors (obesity, hydration, skin thickness, etc.) can potentially affect optimal acoustics (e.g., SONAR—Sound Optimizing dilatioN for Angiogenic Response).

In some embodiments, systems and methods as disclosed herein can also treat venous insufficiency (e.g., via venous collateral formation), acute or chronic pain, neuropathies, rheumatoid or osteoarthritis, cellulitis, osteomyelitis, or chronic wounds, Raynaud's or other vasospastic diseases, peripheral edema including venous stasis and lymphedema, erectile dysfunction in both males and females (e.g., via focused ultrasound to the, e.g., pudendal or clitoral artery), ischemic bowel (e.g., via focused ultrasound to arteries/tissue of the GI tract), or a variety of other indications. In some embodiments systems and methods as disclosed herein can be part of a combination therapy to achieve an unexpectedly synergistic result. In some embodiments, ultrasound systems and methods as disclosed herein could be combined with pharmacologic therapy including antiplatelet therapy such as aspirin or clopidogrel, or anticoagulation therapy such as warfarin, heparin, low-molecular weight heparin, dabigatran, rivaroxaban, apixaban, edoxaban, or other agents such as cilostazol and pentoxifylline, or thrombolytics including tPA, streptokinase, urokinase, and others. In some embodiments, systems and methods disclosed herein can treat or prevent restless legs syndrome, which may have a vascular ischemic component as noted above and thus responds to dopaminergic pharmacotherapy, which can promote vasodilation.

In some embodiments, systems and methods could include a combination of any number of the following modalities (in addition to, or as an alternative to one or more ultrasound transducers) to achieve an unexpectedly synergistic benefit: light energy (e.g., via a laser), magnetic energy such as trans-cranial magnetic stimulation, radiofrequency energy, microwave energy, mechanical energy (e.g., vibration or compression), electrical stimulation, thermal energy (e.g., warming), cooling, hypoxia or hyperoxia, or localized drug delivery. In some embodiments, systems and methods can be used to avoid an interventional procedure such as a bypass procedure, angioplasty, or stenting, or to reduce the risk of restenosis or recurrent ischemia following such procedures. In some embodiments, the systems, such as the machines, devices, and apparatuses described herein, can be in communication with a remote computer, such as a desktop, laptop, tablet, smartphone, wearable device, and/or other computing device, that can be located in a remote location, such as a clinician's and/or physician's office. The physician or clinician can review, from the remote computer, any parameter(s) of the system, clinical results, use of the system, current state of the system, and/or other information regarding the system and use. In some embodiments, the physician or clinician can change parameters of the system, send messages to a display of the system, and/or otherwise manipulate the system with the remote computer.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “positioning a wearable ultrasonic sleeve on a patient's lower extremity” includes “instructing the positioning of a wearable ultrasonic sleeve on a patient's lower extremity.”

Although this disclosure has been described in the context of certain aspects and examples, it will be understood by those skilled in the art that the disclosure extends beyond the specifically disclosed aspects to other alternative aspects and/or uses and obvious modifications and equivalents thereof. In addition, while several variations of the aspects of the disclosure have been shown and described in detail, other modifications, which are within the scope of this disclosure, will be readily apparent to those of skill in the art. It is also contemplated that various combinations or sub-combinations of the specific features and aspects of the aspects may be made and still fall within the scope of the disclosure. For example, features described above in connection with one aspect can be used with a different aspect described herein and the combination still fall within the scope of the disclosure. It should be understood that various features and aspects of the disclosed aspects can be combined with, or substituted for, one another in order to form varying modes of the aspects of the disclosure. Thus, it is intended that the scope of the disclosure herein should not be limited by the particular aspects described above. Accordingly, unless otherwise stated, or unless clearly incompatible, each aspect of this invention may comprise, additional to its essential features described herein, one or more features as described herein from each other aspect of the invention disclosed herein.

It should be emphasized that many variations and modifications may be made to the herein-described implementations, the elements of which are to be understood as being among other acceptable examples. All such modifications and variations are intended to be included herein within the scope of this disclosure. The section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

The various features and processes described herein may be used independently of one another, or may be combined in various ways. Features, materials, characteristics, or groups described in conjunction with a particular aspect, embodiment, implementation, example, etc., are to be understood to be applicable to any other aspect, embodiment, implementation, example, etc., described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing aspects. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination. All possible combinations and sub-combinations are intended to fall within the scope of this disclosure.

Moreover, while operations may be depicted in the drawings or described in the specification in a particular order, such operations need not be performed in the particular order shown or in sequential order, or that all operations be performed, to achieve desirable results. For example, in some implementations, certain acts, events, functions, operations, or method or process blocks of any of the algorithms described herein can be performed in a different sequence than that specifically disclosed, can be added, merged, rearranged, or left out altogether (for example, not all described acts or events are necessary for the practice of the algorithms). Moreover, in certain implementations, acts, events, functions, operations, or method or process blocks can be performed concurrently, for example, through multi-threaded processing, interrupt processing, or multiple processors or processor cores or on other parallel architectures, rather than sequentially. In addition, different tasks or processes can be performed by different machines and/or computing systems that can function together. Other operations that are not depicted or described can be incorporated in the example methods and processes. For example, one or more additional operations can be performed before, after, simultaneously, or between any of the described operations. Furthermore, the features and attributes of the specific aspects disclosed above may be combined in different ways to form additional aspects, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Any process, descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the implementations described herein in which elements or functions may be deleted, executed out of order from that shown or discussed, including substantially concurrently or in reverse order, depending on the functionality involved, as would be understood by those skilled in the art.

Each of the processes, methods, and algorithms described in the preceding sections may be embodied in, and fully or partially automated by, code modules executed by one or more computer systems or computer processors including computer hardware. The code modules may be stored on any type of non-transitory computer-readable medium or computer storage component, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various illustrative logical blocks, modules, and algorithm elements described in connection with the implementations disclosed herein can be implemented as electronic hardware, computer software, or combinations of both. To clearly illustrate this interchangeability of hardware and software, various illustrative components, blocks, modules, and elements have been described herein generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another implementation, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the implementations disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

For purposes of this disclosure, certain aspects, advantages, and novel features are described herein. Not necessarily all such advantages may be achieved in accordance with any particular aspect described herein. Thus, for example, those skilled in the art will recognize that certain aspects may be configured to operate in a manner that achieves or optimizes one advantage or group of advantages as taught herein without necessarily achieving other objects or advantages as may be taught or suggested herein.

As used herein, “system,” “instrument,” “apparatus,” and “device” generally encompass both the hardware (for example, mechanical and electronic) and, in some implementations, associated software (for example, specialized computer programs for graphics control) components.

Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation.

Conjunctive language such as the phrase “at least one of X, Y, and Z,” or disjunctive language such as the phrase “at least one of X, Y, or Z,” unless specifically stated otherwise, is to be understood with the context as used in general to convey that an item, term, and/or the like may be either X, Y, or Z, or any combination thereof (for example, X, Y, and/or Z). For example, the term “or” is used in its inclusive sense (and not in its exclusive sense) so that when used, for example, to connect a list of elements, the term “or” means one, some, or all of the elements in the list. Thus, such conjunctive or disjunctive language is not generally intended to imply that certain implementations require at least one of X, at least one of Y, and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

The term “comprising” as used herein should be given an inclusive rather than exclusive interpretation. For example, a general-purpose computer comprising one or more processors should not be interpreted as excluding other computer components, and may possibly include such components as memory, input/output devices, and/or network interfaces, among others.

While the above detailed description has shown, described, and pointed out novel features as applied to various implementations, it may be understood that various omissions, substitutions, and changes in the form and details of the devices or processes illustrated may be made without departing from the spirit of the disclosure. As may be recognized, certain implementations of the inventions described herein may be embodied within a form that does not provide all of the features and benefits set forth herein, as some features may be used or practiced separately from others. The scope of the present disclosure is not intended to be limited by the specific disclosures of preferred aspects in this section or elsewhere in this specification. The scope of the present disclosure is indicated by the appended claims rather than by the foregoing description, and may be indicated by claims presented in the future. The language of the claims is to be interpreted broadly based on the language employed in the claims and not limited to the examples described in the present specification or during the prosecution of the application, which examples are to be construed as non-exclusive. All changes which come within the meaning and range of equivalency of the claims are to be embraced within their scope.

1. A method of treating calcified vascular tissue, comprising:
providing a therapeutic ultrasound device configured to generate a therapeutic ultrasound acoustic field;
providing a diagnostic imaging ultrasound transducer coupled to or used in conjunction with the therapeutic ultrasound device and configured to display a location of the therapeutic ultrasound acoustic field superimposed on the diagnostic ultrasound image;
positioning the ultrasound transducer proximate a skin surface of a patient;
focusing an acoustic field of the therapeutic ultrasound transducer onto a region of vascular calcium within a vessel of the patient below the skin surface;
generating a therapeutic ultrasound energy waveform, the therapeutic ultrasound energy waveform comprising a therapeutically effective amount of therapeutic ultrasound energy; and
directing the therapeutically effective amount of therapeutic ultrasonic energy toward the region of vascular calcium to stimulate cavitation and shear stress and to remove or soften a calcium deposit within the region of vascular calcium.

2. The method of claim 1, wherein the therapeutic ultrasonic energy has a frequency of 400 kHz to 5 MHz.

3. The method of claim 1, wherein the therapeutic ultrasonic energy has a pulse duration of 5 μs to 100 ms.

4. The method of claim 1, wherein the therapeutic ultrasonic energy has a pulse repetition frequency of 0.1 Hz to 5 kHz.

5. The method of claim 1, wherein the therapeutic ultrasonic energy has a peak negative pressure of 2 MPa to 24 MPa.

6. The method of claim 1, wherein the therapeutic ultrasound transducer comprises an array of ultrasound transducers.

7. The method of claim 6, wherein the array of therapeutic ultrasound transducers is a linear array.

8. The method of claim 1, wherein the therapeutic ultrasound transducer is geometrically focused, and/or electronically focused, and/or comprises electronic and/or mechanical beam steering.

9. The method of claim 1, wherein the therapeutic ultrasound transducer is shaped to treat a longitudinal segment of the vessel.

10. The method of claim 9, wherein the longitudinal segment of the vessel is up to 20 cm long.

11. The method of claim 1, wherein directing the therapeutically effective amount of therapeutic ultrasonic energy toward the region of vascular calcium comprises directing the therapeutically effective amount of therapeutic ultrasonic energy toward the region of vascular calcium for 1 to 120 minutes.

12. The method of claim 1, wherein directing the therapeutically effective amount of therapeutic ultrasonic energy toward the region of vascular calcium comprises directing the therapeutically effective amount of therapeutic ultrasonic energy toward the region of vascular calcium for a plurality of intervals.

13. The method of claim 1, further comprising providing an imaging transducer configured to generate an image of the vessel.

14. The method of claim 13, further comprising generating an image of the region of vascular calcium within the vessel.

15. The method of claim 13, further comprising displaying the image on a display coupled to the therapeutic ultrasound device.

16. The method of claim 13, wherein the therapeutic ultrasound device is further configured to automatically position the ultrasound transducer based on the image.

17. The method of claim 13, wherein the therapeutic ultrasound device is further configured to automatically focus the acoustic field of the ultrasound transducer onto the region of vascular calcium based on the image.

18. The method of claim 13, further comprising providing a microbubble solution to the vessel.

19. The method of claim 1, wherein the vessel comprises an iliac artery, a common femoral artery, a superficial femoral artery, a popliteal artery, an anterior tibial artery, a posterior tibial artery, a subclavian artery, an axillary artery, a brachial artery, a radial artery, an ulnar artery, a renal artery, a carotid artery, a celiac trunk/artery, a superior mesenteric artery, or an inferior mesenteric artery.

20. The method of claim 1, wherein the region of vascular calcium comprises the dorsalis pedis, the posterior tibial, or the pedal arches.

21. The method of claim 1, further comprising performing a secondary therapy on the vessel after therapeutic ultrasound.

22. The method of claim 21, wherein said secondary therapy comprises stenting, drug-coated balloon angioplasty, balloon angioplasty, shockwave atherectomy, orbital atherectomy, or otherwise removing calcium from the vessel.

23. The method of claim 1, further comprising identifying fluid flow within the vessel.
